(19) United States
(12) Patent Application Publication
(10) Pub. No.: US 2013/0337009 A1
(43) Pub. Date: Dec. 19, 2013

(54) CHIMERIC DNA VACCINE COMPOSITIONS AND METHODS OF USE

(71) Applicant: ImmunoGenetix Therapeutics, Inc., (US)

(72) Inventor: Yahia Chebloune, Grenoble (FR)

(73) Assignee: IMMUNOGENETIX THERAPEUTICS, INC., Lenexa, KS (US)

(21) Appl. No.: 13/772,279

(22) Filed: Feb. 20, 2013

Related U.S. Application Data
(60) Provisional application No. 61/601,322, filed on Feb. 21, 2012.

(51) Int. Cl. A61K 39/21; C12N 15/85

(52) U.S. Cl. CPC A61K 39/21; C12N 15/85
USPC 424/208.1; 435/320.1; 424/225.1; 424/229.1

(57) ABSTRACT

The present invention relates to compositions and methods for the prevention and treatment of infectious diseases. In particular, the invention relates to stimulating an immune response in a subject to prevent or treat diseases by administering a DNA vaccine encoding regulatory elements derived from the caprine arthritis encephalitis goat lentivirus genome and at least one immunogenic molecule to the subject. The immunogenic molecules used with the present invention may be capable of stimulating an immune response to any infectious disease causing agent. In particular, the invention is useful for stimulating an immune response to infectious diseases caused by lentiviruses. For instance, the present invention is directed to a DNA vaccine for immunization against HIV.

### SEQUENCE LISTING

A sequence listing in electronic format is being filed together with the application. The content of this sequence listing is incorporated by reference in its entirety.

### FIELD OF THE INVENTION

The present invention relates generally to the field of prophylactic vaccines for generating protection from infectious disease and infection. More specifically, the present invention relates to DNA vaccines capable of stimulating an immune response in a subject. This invention is useful for protection and treatment of infection by acquired immunodeficiency disease causing agents such as Human Immunodeficiency Virus (HIV).

### BACKGROUND OF THE INVENTION

Human Immunodeficiency Virus (HIV) continues to be a worldwide health problem with over 33 million individuals infected. Each year, nearly 3 million people become infected and over 2 million die. Among the infected individuals, there is a small subset of Long-Term Non-Progressors (LTNP) and Elite Suppressors (ES). These individuals carry the virus, but do not develop AIDS. It has been found that some of these individuals have been infected with naturally attenuated HIV-1 variants that harbor mutations in the nef gene (Live-attenuated). The existence of this live-attenuated infection holds promise for derivation of a vaccine against pathogenic HIV.

Attempts at deriving vaccines for immunodeficiency diseases have been met with many challenges. Vaccines derived from simian immunodeficiency viruses have provided reproducible protection in non-human primates. However, these vaccines, derived from pathogenic strains of viruses, caused persistent infections with integration of their provirus into the genome of the treated subject. Further, they were found to retain pathological properties in infants and reversion to pathogenic phenotype in some adult macaques. These problems are related to the replicating and integrating capabilities of this type of vaccine. Therefore, despite their high efficacy at inducing protective immune response, these types of vaccines are excluded from possible use in humans because of the ethical as well as safety issues surrounding their use.

While non-replicating, non-integrating HIV-based DNA vaccines have been developed that induce potent immune responses in rodents, these responses were found to be very weak in primates and required additional heterologous boost with proteins or viruses. For example, several DNA vaccine constructs encoding HIV proteins under a strong enhancer/promoter such as the human cytomegalovirus (CMV) have been developed and used. However, none of the vaccination regimens using these DNA vaccines alone has yet induced potent immune responses that were found to be associated with complete protection in absence of boost with recombinant vectors expressing viral proteins. Efforts have also been made to enhance the immunogenicity of these vaccines by optimizing their delivery, the expression of viral antigens, or targeting a particular compartment in expressing cells. However, these efforts have not provided a vaccine capable of providing protection equivalent to that induced by live-attenuated vaccines, especially when high pathogenic viruses are used for challenge.

The CMV promoter is well known for promoting constitutive gene expression and has been shown to drive the production of infectious particles when used in replacement of the U3 region in the HIV 5′LTR. However, despite a constitutive and high expression efficacy, this promoter was associated with the production of lower infectious titers than the wild-type virus genome, suggesting that this type of promoter is not sufficient to produce a high yield of viral proteins that are efficiently assembled into infectious particles (Bohne J. Schambach Al, Zychlinski D. J. Virol 2007 April; 81(7):3652-6). The use of the CMV promoter does not preserve the regulation of alternative splicing controlled by viral LTRs and leads to an excessive accumulation of multiply spliced, viral RNA genome. Viral LTRs balance gene expression through a controlled alternative splicing process.

Live-attenuated vaccines have been shown to be the most effective vaccines against AIDS in non-human primate models. These vaccines mimic natural infection and induction of immune responses by the host, but they are not considered to be safe due to the associated risk of reversion into pathogenic virus.

Testing of vaccine efficacy generally requires the challenge of a subject with live virus or DNA. It is ethically and practically difficult to attempt preliminary studies using human subjects. The use of model systems for preliminary design and testing of candidate vaccines has been hampered by various species-specific features of lentiviruses. For instance, with HIV the HIV-1 virus is known only to infect certain endangered species of chimpanzees in addition to humans. The feasibility of obtaining sufficient numbers of such endangered animals for full preliminary study of HIV-1 virus vaccines is quite low. It is preferable to use validated analogous animal model systems in such cases.

One analogous model system for HIV-1 has been the SIV_{mac} (Simian Immunodeficiency Virus, macaque) system. SIV infects a variety of simians, including macaques, but the differences between SIV and HIV make SIV of limited use as a potential human vaccine. Further, chimeric SIV-HIV DNA vaccines, which allow study of HIV in an analogous animal model, have potential safety issues when used in humans. In particular, chimeric SIV-HIV DNA vaccines have the potential to recombine with replication-competent HIV causing the DNA vaccine to become replication competent and generate replication competent HIV recombinants.

There remains a need for HIV-vaccines that overcome the risk of host genome integration, replication capabilities, and the potential to undergo recombination that may lead to the emergence of a pathogenic virus. Further, there remains a need for a DNA vaccine that generates an immune response in both humans and an analogous animal model and that lacks the ability to recombine with naturally occurring lentiviruses. The present invention provides a vaccine that solves the above-described problems. The DNA vaccine uses elements from multiple lentiviruses to produce a vaccine that is non-integrating, non-replicating, and not capable of recombination. At the same time, the DNA vaccine is able to induce an immune response similar to that induced by live-attenuated virus.

### SUMMARY OF INVENTION

The present invention is directed to a DNA vaccine for immunization against infectious disease causing agents. The invention comprises a DNA molecule that has a sequence encoding at least one immunogenic molecule capable of stimulating an immune response in a subject.

One embodiment includes a method of immunizing a subject against an infectious disease by administering a DNA vaccine comprising a DNA composition of the invention to the subject. It is contemplated that a DNA vaccine including DNA compositions of the invention may be used for treatment or immunization of a subject against any of the diseases described herein and for any newly discovered diseases from which immunogenic molecules may be derived. Herein, the present invention provides DNA vaccine compositions that may be used for treatment or immunization of a subject against acquired immunodeficiency diseases. In particular, such compositions may be used in humans for treatment or protection from HIV including HIV-1 and HIV-2 as well as variants thereof; in simians for treatment or protection from SIV; and felines for treatment or protection from FIV, as well as other species and species-specific immunodeficiency viruses known in the art.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF THE SEQUENCES

SEQ ID NO: 1 encodes the CAL-Δ4 DNA vaccine construct.

SEQ ID NO: 2 encodes the CAEV promoter sequence (5′LTR).

SEQ ID NO: 3 encodes the SV40 polyadenylation sequence (SV40 polyA).

SEQ ID NO: 4 encodes sequence deleted from SIV 3′LTR of CAL-Δ4 DNA vaccine construct.

SEQ ID NO: 5 encodes SIV gag and pol gene coding sequence (including rt and int genes) removed from the immunogenic molecule sequence to make the chimeric SHIV immunogenic molecule sequence.

SEQ ID NO: 6 encodes sequence of the first 472 nucleotides of SIV vif gene removed from the immunogenic molecule sequence to make the chimeric SHIV immunogenic molecule sequence.

SEQ ID NO: 7 encodes HIV gag and pol gene coding sequence used to replace SIV gag and pol gene sequence to make the chimeric SHIV immunogenic molecule sequence.

SEQ ID NO: 8 encodes the CAL-Δ4 DNA vaccine construct contained in an expression vector for cloning purposes.

SEQ ID NO: 9 encodes the sequence of a chimeric immunogenic molecule sequence including the int gene and comprising regulatory elements of CAEV (CAL-LTR+INT).

SEQ ID NO: 10 encodes the sequence of a chimeric immunogenic molecule sequence comprising regulatory elements of CAEV (CAL-LTR).

SEQ ID NO: 11 encodes the sequence of a chimeric immunogenic molecule sequence comprising regulatory elements of CAEV (CAL-LTR).

SEQ ID NO: 12 encodes the sequence of a chimeric immunogenic molecule sequence comprising regulatory elements of CAEV (CAL-LTR).

SEQ ID NO: 13 encodes the sequence of a chimeric immunogenic molecule sequence comprising regulatory elements of CAEV (CAL-LTR).

SEQ ID NO: 14 encodes the 3′LTR of CAEV.

### BRIEF DESCRIPTION OF THE FIGURES

The application file contains at least one photograph executed in color. Copies of this patent application publication with color photographs will be provided by the Office upon request and payment of the necessary fee. The following drawings form part of the present specification and are included to further demonstrate certain aspects of the present invention. The invention may be better understood by reference to one or more of these drawings in combination with the detailed description of specific embodiments presented herein.

FIG. 1 illustrates DNA vaccines of the invention. FIG. 1A depicts the Δ4SHIVKU2 DNA vaccine construct, that is regulated by the SIV 5′LTR and SV40 Poly A termination sequence. FIG. 1B depicts the CAL-Δ4 DNA vaccine construct, that is regulated by the CAEV 5′LTR and SV40 Poly A termination sequence. FIG. 1C depicts the CAL-LTR DNA vaccine construct, that is regulated by the CAEV 5′LTR and 3′LTR.

FIG. 2 shows the detection of HIV antigens produced by the CAL-Δ4 DNA vaccine genome by ELISA (FIG. 2A) and radio-immunoprecipitation (FIG. 2B).

FIG. 3 graphically illustrates that mice immunized with the CAL-Δ4 DNA vaccine developed immune responses against Gag, Env, Tat, Rev, and Nef.

FIG. 4 shows the vast majority of the vaccine-induced T cells of mice immunized with the CAL-Δ4 DNA vaccine did not produce detectable IFN-γ upon re-stimulation.

FIG. 5 shows the vast majority of the vaccine-induced T cells of rhesus macaques immunized with the CAL-Δ4 DNA vaccine did not produce detectable IFN-γ upon re-stimulation.

FIG. 6 shows a comparison of immune responses detected in mice treated with HIV DNA vaccines. FIG. 6A graphically illustrates a comparison of the number of IFN-γ producing cells activated in response to mice immunized with Δ4SHIVkU2, CAL-SHIV, or SHIVKU2 DNA vaccines (SHIVKU2 is regulated by the SIV 5′LTR and 3′LTR). FIG. 6B graphically illustrates a comparison of the percentage of HIV-specific CD3+ T cells secreting IFN-γ or IL-2 cytokine after immunization with CA-LTR or SHIV2 in response to antigens Gag, Env, or TRN.

FIG. 7 shows T cell responses of mice humanized with human peripheral blood mononuclear cells (PBMCs) and immunized with DNA vaccine. FIG. 7A shows the initial gating on live human lyphocytes EMA- CD3+CD4+ (blue) or CD8+(orange). FIG. 7B shows T cell responses to Gag, Env, or TRN after 16 hours and gated on CD3+ CD8+. FIG. 7C shows T cell responses to Gag, Env, or TRN after 16 hours and gated on CD3+CD4+.

FIG. 8 shows IFN-γ ELISPOT T cell responses to Gag, Env, TRN, and Pol antigens of mice humanized with human PBMCs and vaccinated with a DNA vaccine. FIGS. 8A-E graphically illustrate the T cell responses of cells isolated from individual mice BX80, BX83, BX72, BX84, and BX78, respectively.

FIG. 9 shows primary CD8+ T cell responses to Gag antigen exposure for 16 hours and 5 days. FIG. 9A shows the initial gating on live lymphocytes EMA-CD3+CD8+ (orange) or CD4+ (blue). FIG. 9B shows T cell responses of cells isolated from mice BX78 and BX72 pre-immunization and during the primary expansion phase (week 2-4 and week 6 after immunization). FIG. 9C shows T cell responses of cells isolated from mice BX80 and BX84 pre-immunization and during the primary expansion phase (week 2-4 and week 6 after immunization).

FIG. 10 shows contraction and memory CD8+ T cell responses to Gag antigen after 16 hours and 5 days of exposure. FIG. 10A shows the initial gating on live lymphocytes EMA-CD3+CD8+ (orange) or CD4+ (blue). FIG. 10B shows T cell responses of cells isolated from mice BX78 and BX72 during the contraction phase (Weeks 8-14 post immunization) and the reemergence phase (weeks 18-26 post immunization). FIG. 10C shows T cell responses of cells from mice BX80 and BX84 during the contraction phase and reemergence phase.

FIG. 11 shows phenotyping of CD8+ T cells. FIG. 11A shows the initial gating on live lymphocytes EMA-CD3+CD8+ (orange) or CD4+ (blue) of cells isolated from mouse BX73. FIG. 11B shows T cell responses to TRN antigen 8 weeks after immunization with a DNA vaccine. FIG. 11C shows T cell responses to TRN antigen four weeks after immunization.

FIG. 12 shows CD4+ and CD8+ T cell responses 20 weeks after immunization with a DNA vaccine. The CD8+ T cell responses are graphically illustrated for cells isolated from individual mice BX78 (FIG. 12A), BX72 (FIG. 12B), BX80 (FIG. 12C), and BX84 (FIG. 12D). The CD4+ T cell responses are graphically illustrated for cells isolated from individual mice BX78 (FIG. 12E), BX72 (FIG. 12F), BX80 (FIG. 12G), and BX84 (FIG. 12H).

FIG. 13 shows CD4+ and CD8+ T cell responses 20 weeks after immunization with a DNA vaccine. The CD8+ T cell responses are graphically illustrated for cells isolated from individual mice BX83 (FIGS. 13A and 13B), and BX73 (FIG. 13C). The CD4+ T cell responses are graphically illustrated for cells isolated from individual mice BX83 (FIGS. 13D and 13E) and BX73 (FIG. 13F).

### DETAILED DESCRIPTION OF THE INVENTION

In accordance with the present invention, a composition that is capable of treating or preventing infectious disease as well as methods of use have been discovered. In particular, the present invention relates to novel nucleic acid compositions and uses thereof. The nucleic acid sequences of the invention may be used to stimulate an immune response in a subject to prevent or treat infectious disease with significantly enhanced safety over other methods of prevention and treatment known in the art. The invention is particularly useful in the treatment and prevention of infectious diseases caused by lentiviruses, such as immunodeficiency diseases. The compositions and methods of using the composition are discussed in more detail below.

### I. Compositions

Compositions useful in this invention, such as those described below, are generally able to be used as a treatment therapy or preventative therapy for infectious diseases without integrating into the host subject's genome. Further, such compositions are generally unable to produce pathogenic recombinants.

### A. Nucleic Acids

The present invention provides nucleic acid molecules useful for the treatment of infectious diseases, such as immunodeficiency disease causing agents. The invention further provides nucleic acid molecules included in a DNA vaccine construct. The DNA vaccine construct includes immunogenic molecules and regulatory elements. The invention further provides nucleic acid molecules, vectors, and host cells (in vitro, in vivo, or ex vivo) which contain the DNA vaccine construct of the invention.

### 1. Immunogenic Molecules

In some embodiments, the DNA vaccine construct of the invention encodes at lest one immunogenic molecule. The DNA vaccine construct may encode about 1, 2, 3, 4, 5, 6, 7, 8, 9, 10, 11, 12, 13, 14, 15, 16, 17, 18, 19, 20, 21, 22, 23, 24, 25 or more immunogenic molecules. Such DNA vaccine constructs, encoding more than one immunogenic molecule, may also encode linker sequences between each immunogenic molecule.

Immunogenic molecules of the invention may be any immunogen, antigen, peptide, protein, or small molecule. Suitable immunogenic molecules are those that are known or expected to illicit a desired immune response sufficient to yield a therapeutic or protective effect when used with the compositions of the present invention. Immunogenic molecules may be any antigen currently used to produce vaccines and those yet to be discovered in the art.

One aspect of the present invention is directed to DNA vaccine constructs that encode immunogenic molecules capable of stimulating an immune response against immunodeficiency disease causing agents, such as HIV, SIV, FIV, and variants thereof, as well as others known in the art. In some embodiments, at least one immunogenic molecule is selected among gag, pol, vif, vpx, vpr, nef, tat, rev, vpu, env, pro, int, rt, or combinations thereof. In some embodiments, the immunogenic molecules are the gag, pro, vpx, vpr, nef, and tat proteins of immunodeficiency disease causing agents, such as HIV, SIV, FIV, HIV-1, HIV-2 and others known in the art. In other embodiments, the immunogenic molecules are the gag, pro, vpx, vpr, and nef proteins of immunodeficiency disease causing agents. The immunogenic molecules may be of any genetic clade of immunodeficiency virus or may be synthetic sequence derived from conserved regions of several genetic clades. Further, the immunogenic molecules may be of any species, such as HIV, SIV, FIV, CAEV and others known in the art, as well as combinations thereof.

In one embodiment, the immunogenic molecules are nucleic acid sequences encoding the gag, pro, vpx, vpr, nef, and tat proteins of immunodeficiency disease causing agents. In another embodiment, the immunogenic molecules are nucleic acid sequences encoding the gag, pro, vpx, vpr, and nef, proteins of immunodeficiency disease causing agents. In one embodiment, the nucleic acid sequences encode the full protein sequence. In another embodiment, the nucleic acid sequences encode partial protein sequence. In another embodiment, the nucleic acid sequences encode the full protein sequence of gag, pro, vpx, vpr, and nef. In another embodiment, the nucleic acid sequences encode the full protein sequence of gag, pro, vpx, vpr, and nef proteins and encode a partial sequence of tat protein. In another embodiment, the nucleic acid sequences encode the full protein sequence of gag, pro, vpx, vpr, and nef proteins and encode a partial sequence of tat, reverse transcriptase (rt), integrase (int), and viral infectivity factor (vif) proteins. In one embodiment, the nucleic acid sequence is not capable of producing a protein capable of its normal bioactive activity.

### 2. Regulatory Elements

In some embodiments, the DNA vaccine construct encodes at least one regulatory sequence. The regulatory sequence is operatively linked to the immunogenic molecules. Suitable regulatory sequences include those encoding expression regulators, such as promoters and 5′LTRs; termination sequences, such as 3′LTRs or poly(A) sequences; and any regulatory sequence known in the art or yet to be discovered. The regulatory sequence may be any sequence known in the art or derived therefrom. In some embodiments, the DNA vaccine construct encodes an expression regulator. In some embodiments, the DNA vaccine construct encodes an expression regulator and termination sequences. In some embodiments, the expression regulator is a promoter. In other embodiments, the expression regulator is a 5′LTR.

In one embodiment, the regulatory sequence operatively linked to the immunogenic molecule of interest is derived from caprine arthritis encephalitis lentivirus (CAEV) DNA sequence. Preferably, the regulatory sequence is derived from a CAEV regulatory sequence, such as a promoter or termination sequence. More preferably, the regulatory sequence is derived from the CAEV 5′ LTR or 3′LTR sequence or combination thereof. The CAEV regulatory sequence is provided in SEQ ID NO: 2 or SEQ ID NO: 14. In some embodiments the sequence for CAEV 5′LTR is identical or complementary to that of CAEV 3′LTR. A suitable regulatory sequence includes sequences that hybridize under high stringency conditions to the regulatory sequence regions of the sequences contained herein (SEQ ID NO: 1-14, preferably SEQ ID NO: 2 or SEQ ID NO: 14), such as those that are homologous, substantially similar, or identical to the nucleic acids of the present invention. Homologous nucleic acid sequences will have a sequence similarity of at least about 50%, 55%, 60%, 65%, 70%, 75%, 80%, 85%, 90%, 91%, 92%, 93%, 94%, 95%, 96%, 97%, 98%, 99%, or 100% to regulatory portions of SEQ ID NO: 1-14, SEQ ID NO: 2, SEQ ID NO: 14 or the respective complementary sequence thereof. This promoter drives the expression of immunogenic molecules present in the DNA vaccine. Those skilled in the art will recognize that alternative embodiments of this invention may substitute other functional promoter sequences that will also drive expression of the desired immunogenic molecules. However, an advantage of using the CAEV derived regulatory sequence is that the potential for recombination events to produce replication and integration competent recombinants is eliminated.

### 3. Modifications

Mutant nucleotides of the DNA molecules of the invention may be used, so long as mutants include nucleic acid sequences that encode peptides capable of stimulating an immune response or regulating expression of immunogenic molecules as described herein. The DNA sequence or protein product of such a mutation will usually differ by one or more nucleotides or amino acids. The sequence changes may be substitutions, insertions, deletions, or a combination thereof. Techniques for mutagenesis of cloned genes are known in the art. Methods for site specific mutagenesis may be found in Gustin et al., Biotechniques 14:22, 1993; Barany, Gene 37:111-23, 1985; Colicelli et al., Mol. Gen. Genet. 199:537-9, 1985; and Sambrook et al., Molecular Cloning: A Laboratory Manual, CSH Press 1989, pp. 15.3-15.108 and all incorporated herein by reference. In summary, the invention relates to nucleic acid sequences capable of stimulating an immune response in a subject and variants or mutants thereof. Also, the invention encompasses the intermediatary RNAs encoded by the described nucleic acid sequences and that translates into an antigenic peptide of the invention, as well as the resultant antigenic peptide.

One skilled in the art will recognize that genetic variants derived from infectious disease causing agents may easily be constructed using DNA mutagenesis and cloning techniques known in the art with the present invention. This may be particularly advantageous when constructing DNA vaccines against high genetically-variable agents such as HIV. For instance, one skilled in the art will recognize that the genetic alterations that occur in evolving HIV can easily be introduced into the compositions and methods herein.

Importantly, the DNA molecules of the present invention have been disrupted functionally such that the ability of these molecules to encode functional proteins important in pathogenicity is removed. For instance, in the embodiments directed to acquired immunodeficiency diseases the vif, int and rt genes of the DNA vaccine are disrupted. Other embodiments functionally disrupt the rt gene. Some embodiments functionally disrupt the int gene. It is anticipated that the DNA can be disrupted functionally by inserting or deleting at least one nucleotide such that the number of nucleotides in the altered sequences differs with respect to the unaltered sequences. It is also anticipated that the DNA encoding immunogenic molecules can be disrupted functionally by substituting one or more nucleotides that encode functional amino acids with one or more distinct nucleotides that encode non-functional amino acids. Preferably, the functional disruption of the DNA encoding immunogenic molecules occurs via deletion of at least one of the rt, int, and vif genes or combinations thereof.

Another important aspect of this invention is that it provides for DNA vaccines that disrupt the 3′ LTR sequences that enable undesirable integration of DNA sequences into the host genome. Function of the 3′ LTR can also be abolished by substituting functional nucleotides with distinct non-functional nucleotides. The deleted 3′ LTR region is preferably replaced with an SV40 polyadenylation sequence or 3′LTR derived from CAEV or other viral source except HIV or SIV. Those skilled in the art will recognize that polyadenylation sites derived from a variety of sources other than SV40 may also be used as substitutes for the 3′ LTR sequences.

Yet, another important aspect of this invention provides for DNA compositions capable of stimulating an immune response against HIV including nucleic acid sequences that hybridize under high stringency conditions to SEQ ID NO: 1-14. Suitable DNA compositions include nucleic acid sequences such as those that are homologous, substantially similar, or identical to the nucleic acids of the present invention. Homologous nucleic acid sequences will have a sequence similarity of at least about 50%, 55%, 60%, 65%, 70%, 75%, 80%, 85%, 90%, 91%, 92%, 93%, 94%, 95%, 96%, 97%, 98%, 99%, or 100% to SEQ ID NO: 1 or the respective complementary sequence. Sequence similarity may be calculated using a number of algorithms known in the art, such as BLAST, described in Altschul, S. F., et al., J. Mol. Biol. 215:403-10, 1990. The nucleic acids may differ in sequence from the above-described nucleic acids due to the degeneracy of the genetic code. In general, a reference sequence will be 18 nucleotides, more usually 30 or more nucleotides, and may comprise the entire nucleic acid sequence of the composition for comparison purposes.

Nucleotide sequences that can hybridize to SEQ ID NO: 1-14 are contemplated herein. Stringent hybridization conditions include conditions such as hybridization at 50° C. or higher and 0.1×SSC (15 mM sodium chloride/1.5 mM sodium citrate). Another example is overnight incubation at 42° C. in a solution of 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA, followed by washing in 0.1×SSC at about 65° C. Exemplary stringent hybridization conditions are hybridization conditions that are at least about 80%, 85%, 90%, or 95% as stringent as the above specific conditions. Other stringent hybridization conditions are known in the art and may also be employed to identify homologs of the nucleic acids of the invention (Current Protocols in Molecular Biology, Unit 6, pub. John Wiley & Sons, N.Y. 1989).

### II. Methods of Use

An object of the present invention is to provide DNA compositions, DNA vaccines, and methods that provide either protective immunity to uninfected subjects or therapeutic immunity to infected subjects. As such, the compositions of the invention may be used to prophylactically immunize a subject or used to treat a subject. Both types of methods include administering a DNA composition of the invention to a subject.

### A. Conditions for Use

Conditions that would benefit from use of the DNA vaccine compositions may include any condition or disease that is caused by or related to infection of a subject with a lentivirus. For instance, exemplary conditions that may benefit from use of the DNA vaccine compositions include those conditions caused by or related to infection of a subject with an immunodeficiency disease causing agent such as HIV, SIV, FIV, HIV-1, HIV-2 or any other virus known in the art or yet to be discovered, and variants thereof.

### B. Delivery Means and Routes

There are many ways of presenting the DNA compositions of the present invention to a subject. DNA vaccines may consist of naked DNA plasmid encoding the immunogenic molecule, bacterial vectors, replicon vectors, live attenuated bacteria, DNA vaccine co-delivery with live attenuated vectors, and viral vectors for expression of heterologous genes as well as other methods known in the art or yet to be discovered. In the case of naked DNA replicon vectors, a mammalian expression plasmid serves as a vehicle for the initial transcription of the replicon. The replicon is amplified within the cytoplasm, resulting in more abundant mRNA encoding the immunogenic molecule such that initial transfection efficiency may be less important for immunogenicity. Live attenuated viral vectors (i.e. recombinant vaccinia, adenovirus, avian poxvirus, poliovirus, and alphavirus virion vectors) have been successful in inducing cell-mediated immune response and may be used as well. Attenuated bacteria may also be used as a vehicle for DNA vaccine delivery. Examples of suitable bacteria include S. enterica, S. typmphimurium, Listeria, and BCG. The use of mutant bacteria with weak cell walls can aid the exit of DNA plasmids from the bacterium.

The DNA compositions of the present invention may be administered, or inoculated, to a subject as naked nucleic acid molecules in a physiologically compatible solution such as water, saline, Tris-EDTA buffer, or in phosphate buffered saline. They may also be administered in the presence of substances, such as facilitating agents and adjuvants that have the capability of promoting nucleic acid uptake or recruiting immune system cells to the site of inoculation.

Those of skill in the art will understand that the compositions disclosed herein may incorporate known injectable, physiologically acceptable sterile solutions. For preparing a ready-to-use solution for parenteral injection or infusion, aqueous isotonic solutions, e.g. saline or plasma protein solutions, are readily available. In addition, the compositions of the present invention can include diluents, isotonic agents, stabilizers, or adjuvants.

The medium in which the DNA vector is introduced should be physiologically acceptable for safety reasons. Suitable pharmaceutical carriers include sterile water, saline, dextrose, glucose, or other buffered solutions. Included in the medium can be physiologically acceptable preservatives, stabilizers, diluents, emulsifying agents, pH buffering agents, viscosity enhancing agents, colors, etc.

DNA uptake may be improved by the use of adjuvants. Synthetic polymers (i.e. polyamino acids, co-polymers of amino acids, saponin, paraffin oil, and muramyl dipeptide) and liposomal formulations may be added as adjuvants to the vaccine formulation to improve DNA stability and DNA uptake by the subject and may decrease the dosage required to induce an effective immune response. Regardless of route, adjuvants may be administered before, during, or after administration of the nucleic acid.

DNA uptake may also be improved in other ways known in the art as well. For example, DNA uptake via intramuscularly (IM) delivery of vaccine may be improved by the addition of sodium phosphate to the formulation. Increased DNA uptake via IM delivery may also be accomplished by electrotransfer. Co-injection of cytokines, ubiquitin, or co-stimulatory molecules may also help improve immune induction. The immunogenic molecules of the invention may also be fused with cytokine genes, helper epitopes, ubiquitin, or signal sequences to enhance an immune response. Fusions may also be used to aid in targeting certain cells.

Once the DNA vaccine is delivered, the nucleotide sequences are taken up into the cells of the subject, which then express the nucleotide sequences as protein. The protein is processed and presented in the context of self-major histocompatibility (MHC) class I and class II molecules. The subject then develops an immune response against the encoded immunogenic molecule. To improve the effectiveness of the vaccine, multiple injections may be used for therapy or prophylaxis over extended periods of time.

DNA vaccine compositions may be administered to a subject by a number of methods. Suitable methods of administration include any method known in the art or yet to be discovered. Exemplary administration methods include, without limitation, intradermal, intravenous, intraocular, intratracheal, intratumoral, oral, rectal, topical, intramuscular, intraarterial, intrahepatic, intrathoracic, intrathecal, intracranial, intraperitoneal, intrapancreatic, intrapulmonary, topical, or subcutaneously. Without limiting the scope of administration methods, examples of using various administration methods follow. For example, administration methods may include DNA tattooing; dermal patch delivery; nanoparticle-associated delivery; DNA painting on stripped skin; use of vesicular systems such as liposomes, niosomes, ethosomes and transfersomes; particle-mediated gene gun using microparticles; bacteria delivery systems such as use of Salmonella typhi, Listeria monocytogenes, Shigella flexneri, Yersinia enterocolitica, E. coli and others known in the art; chemical and physical augmentation; electroporation; electropermeabilization; iontophoresis; sonophoresis; chemical permeation enhancers and microneedles; ulstrasound; magnetically and electrically mediated physical methods of gene transfer; and other methods known in the art or yet to be discovered.

### C. Dosage

DNA vaccine compositions of the invention are typically administered to a subject in an amount sufficient to provide a benefit to the subject. This amount is defined as a “therapeutically effective amount.” The therapeutically effective amount will be determined by the efficacy or potency of the particular composition, the duration or frequency of administration, and the size and condition of the subject, including that subject's particular treatment response. Additionally, the route of administration should be considered when determining the therapeutically effective amount. It is anticipated that the therapeutically effective amount of a DNA vaccine composition of the invention will range from about 0.1 μg/kg to 1 mg/kg of total nucleic acid. Suitable doses include from about 5 μg/kg-500 mg/kg of total DNA, 10 μg/kg-250 μg/kg of total DNA, or 10 μg/kg-170 μg/kg of total DNA. In one embodiment, a human subject (18-50 years of age, 45-75 kg) is administered 1.2 mg-7.2 mg of DNA. “Total DNA” and ‘total nucleic acid” refers to a pool of nucleic acids encoding distinct immunogenic molecules. For example, a dose of 50 mg of total DNA encoding 5 different immunogenic molecules can have 1 mg of each molecule. DNA vaccines may be administered multiple times, such as between about 2-6 times. In an exemplary method, 100 μg of a DNA composition is administered to a human subject at 0, 4, and 12 weeks (100 μg per administration).

### D. Methods of Treating Subjects

In one embodiment, the DNA vaccine compositions described herein may be used in methods of treating subjects infected with infectious disease causing agents. In another embodiment, the DNA vaccine compositions described herein may be used in methods of treating subjects not infected with infectious disease causing agents. In one embodiment, the DNA vaccine compositions may be used to treat subjects infected with immunodeficiency disease causing virus. In another embodiment, the DNA vaccine compositions may be used to prevent immunodeficiency disease causing virus infection of a subject. In another embodiment, the DNA vaccine compositions may be used to alleviate conditions caused by, or related to immunodeficiency disease causing virus infection.

The DNA vaccine compositions may be administered to a subject in a single dose or multiple doses. A dosing regimen, either single or multiple dose, may be followed with a booster dose. The amount of time a booster dose may follow a dosing regimen composition depends upon the efficacy of the dosing regimen.

In other embodiments, subjects being administered DNA vaccine compositions of the invention may also be administered combination therapies, in which additional treatments are used. Such additional treatments include therapeutic treatments known in the art, or yet to be discovered, that provide a benefit to the subject. For example, a subject undergoing DNA vaccination against HIV may be administered HIV therapeutics such as anti-retroviral drugs, immunomodulating agents, ribozyme therapies, RNA-based anti-HIV gene genetic therapies, and aptamer therapies. The additional therapeutics may be administered individually, sequentially, or in combination with other therapeutics or the DNA vaccine composition.

Suitable HIV therapeutics include those known in the art as well as those yet to be discovered. Exemplary HIV therapeutics include, without limitation, anti-retroviral drugs, immunomodulating agents, ribozyme therapies, RNA-based anti-HIV gene genetic therapies, and aptamer-based therapies.

A variety of antiretroviral drugs can be used for HIV/AIDS treatment. Antiretroviral (ARV) drugs are broadly classified by the phase of the retrovirus life-cycle that the drug inhibits: (1) Entry inhibitors, also called or fusion inhibitors, including but not limited to maraviroc and enfuvirtide, which interfere with binding, fusion and entry of HIV-1 to the host cell by blocking one of several targets; (2) CCR5 receptor antagonists including maraviroc (Pfizer), aplaviroc (GSK) and vicriviroc (Schering-Plough), which bind to the CCR5 receptor on the surface of the T-Cell and block viral attachment to the cell; (3) Nucleoside reverse transcriptase inhibitors (NRTI), examples of which include Abacavir (Ziagen), adefovir dipivoxil [bis(POM)-PEMA], didanosine (ddI), emtricitabine, lamivudine, lobucavir (BMS-180194), lodenosine (FddA), stavudine (d4t), tenofovir (Truvada), zalcitabine (ddC), zidovudine (Combivir), and 9-(2,3-dideoxy-2-fluoro-b-D-threo-pentofuranosyl)adenine, which inhibit reverse transcription by being incorporated into the newly synthesized viral DNA strand as nucleotides analogs; (4) Non-Nucleoside and nucleotide reverse transcriptase inhibitors (NNRTI), examples of which include efavirenz (Sustiva), etravirine (Intelence), delaviradine (BHAP, U-90152), and nevirapine (Viramune), which inhibit reverse transcriptase by binding to the enzyme; (5) Protease inhibitors (PIs), examples of which include atazanavir (Reyataz), darunavir (Prezista), fosamprenavir (Lexiva), indinavir (MK-639), nelfnavir (AG-1343), ritonavir (Norvir), and saquinavir (Ro 31-8959), which target viral assembly by inhibiting the activity of protease; (6) Integrase inhibitor such as raltegravir (Merck & Co.) inhibits the enzyme integrase, which is responsible for integration of viral DNA into the DNA of the infected cell; and (7) Maturation inhibitors such as IFN-α, bevirimat and vivecon, which blocks the conversion of the polyprotein into the mature capsid protein and the virions released consist mainly of non-infectious particles. In one embodiment, the DNA vaccine composition provided herein is administered in combination with one or more immunomodulating agents and antiretroviral drugs for HIV/AIDS treatment. In one embodiment, the DNA vaccine composition provided herein is administered with any of the above illustrated antiretroviral drug for HIV/AIDS treatment.

Since drug resistance tends to develop during the treatment with any of the antiretroviral drugs, those agents are often administered in combinations. The therapeutic combinations usually comprise two NRTIs and one NNRTI and/or protease inhibitor. In one embodiment, the DNA vaccine composition provided herein is administered with any antiretroviral drug therapeutic combinations for HIV/AIDS treatment.

Sometimes, treatment of HIV/AID uses immunomodulating agents to limit the hyper-elevated state of immune system activation is combined with one or more above mentioned antiretroviral drugs. In one embodiment, the DNA vaccine composition provided herein is administered in combination with one or more immunomodulating agents and antiretroviral drugs for HIV/AIDS treatment.

Ribozyme therapy is also a choice for HIV/AIDS therapy. It uses engineered trans-cleaving ribozymes to cleave specific sequences by mutation of the substrate recognition sequences flanking the cleavage site sequence, and thus can be utilized to remove HIV gene such as U5, pol from the genome to achieve HIV replication inhibition. In one embodiment, the DNA vaccine composition provided herein is administered in combination with one or more engineered trans-cleaving ribozymes, or vectors expressing the trans-cleaving ribozymes, for HIV/AIDS treatment.

RNA-based anti-HIV gene genetic therapies are also among the various HIV/AIDS treatments, which inhibit viral replication via RNA interference. Anti-HIV gene siRNA (small interference RNA) or shRNA (short hairpin) may be engineered for sequence specific mRNA degradation. In addition, long antisense oligonucleotides may be designed to bind to mRNA of a HIV gene and trigger degradation of mRNA through an RNase H dependent pathway or block ribosome binding, and thus inhibiting gene expression. The HIV gene may be targeted include but not limited to HIV env, U1 and trans-activation response (TAR) elements. In one embodiment, the DNA vaccine composition provided herein is administered in combination with one or more anti-HIV gene molecules, or vectors expressing the antisense RNAs, for HIV/AIDS treatment.

Further, aptamers may be used for HIV/AIDS treatment as well. Aptamers are single-stranded RNA or DNA molecules that can bind proteins with high affinity as a decoy. These molecules, normally 15 to 40 bases long, can be used as decoys to bind viral proteins or as vehicles for targeted delivery of siRNAs. A lentiviral vector may be used to express such aptamer, which targets TAR and other viral protein key to virus replication. In one embodiment, the DNA vaccine composition provided herein is administered in combination with one or more aptamers, or aptamer expressing vectors, for HIV/AIDS treatment.

### III. Kits

The present invention provides articles of manufacture and kits containing materials useful for treating the conditions described herein. The article of manufacture may include a container of a compound as described herein with a label. Suitable containers include, for example, bottles, vials, and test tubes. The containers may be formed from a variety of materials such as glass or plastic. The container holds a composition having a DNA vaccine which is effective for treating or preventing HIV infection. The label on the container may indicate that the composition is useful for treating specific conditions and may also indicate directions for administration.

### DEFINITIONS

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

“Administering” or the “administration of” a composition of the invention means delivery of a composition of the invention to a subject by any accepted means in the art. Such appropriate means of administration include intravenous, intra-arterial, intraperitoneal, intramuscular, subcutaneous, intrapleural, topical, or by inhalation. The appropriate means of administering a composition of the invention to a subject will be dependent upon the specific objective to be achieved (e.g. therapeutic, diagnostic, preventative) and the targeted cells, tissues, or organs.

Herein, an “adjuvant” or “adjuvants” can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), non-metabolizable oil, mineral and/or plant/vegetable and/or animal oils, polymers, carbomers, surfactants, natural organic compounds, plant extracts, carbohydrates, water-in-oil emulsion, oil-in-water emulsion, and water-in-oil-in-water emulsion. The emulsion can be based in particular on light liquid paraffin oil (European Pharmacopeia type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di (caprylate/caprate), gly ceryl tri-(caprylate/caprate) or propylene glycol dioleate; or esters of branched fatty acids or alcohols, in particular isostearic acid esters. The oil is used in combination with emulsifiers to form the emulsion. The emulsifiers are preferably nonionic surfactants, in particular esters of sorbitan, mannide (e.g. anhydromannitol oleate), glycol, polyglycerol, propylene glycol, and oleic, isostearic, ricinoleic or hydroxystearic acid, which are optionally ethoxylated, and polyoxypropylene-polyoxyethylene copolymer blocks, in particular the Pluronic products, especially L121. (See Hunter et al., The Theory and Practical Application of Adjuvants (Ed. Stewart-Tull, D. E. S.), John Wiley and Sons, NY, pp 51-94 (1995) and Todd et al., Vaccine 15:564-570 (1997).

The term “construct” refers to a nucleotide sequence that is to be expressed from a vector, for example, the nucleotide sequence encoding immunogenic molecules. In general, a construct comprises a nucleotide sequence inserted into a vector which in some embodiments provides regulatory sequences for expressing the nucleotide sequence. In other embodiments, the nucleotide sequence provides the regulatory sequences for its expression. In further embodiments, the vector provides some regulatory sequences and the nucleotide sequence provides other regulatory sequences. For example, the vector can provide a promoter for transcribing the nucleotide sequence and the nucleotide sequence provides a transcription termination sequence. Suitable regulatory sequences include, but are not limited to, enhancers, transcription termination sequences, kozak sequences, splice acceptor and donor sequences, introns, ribosome binding sequences, and poly(A) addition sequences. The term “vector” refers to some means by which DNA fragments can be introduced into a host organism or host tissue. There are various types of vectors including plasmid, viruses (including adenovirus), artificial chromosomes, bacteriophages, cosmids, and episomes, as well as others known in the art. Vectors may be useful in propagating, targeting, or transferring DNA constructs. In some embodiments, vectors include elements necessary for propagating, targeting, or transferring DNA constructs. Such elements include, without limitation, origins of replication, selectable markers, multiple cloning sites, bacteria resistance, bacteria expression, and regulatory sequences, as well as other elements known in the art or yet to be discovered.

“Diluents”, as used herein, can include water, saline, dextrose, ethanol, glycerol, and the like. “Isotonic agents” can include sodium chloride, dextrose, mannitol, sorbitol, and lactose, among others. “Stabilizers” include albumin and alkali salts of ethylendiamintetracetic acid, among others.

Herein, “effective dose” means, but is not limited to, an amount of a composition of the invention that elicits, or is able to elicit, an immune response that yields a reduction of clinical symptoms in a subject to which the antigen is administered.

An “immunogenic molecule” means a recombinant protein, native protein, or artificial small molecule that stimulates an immune response in a subject. Preferably, an immunogenic molecule does not adversely affect a subject when administered.

An “immune response” or “immunological response” means, but is not limited to, the development of a cellular and/or antibody-mediated immune response to the composition or vaccine of interest. Usually, an immune or immunological response includes, but is not limited to, one or more of the following effects: the production or activation of antibodies, B cells, helper T cells, suppressor T cells, and/or cytotoxic T cells, directed specifically to an antigen or antigens included in the composition or vaccine of interest. Preferably, the subject will display either a therapeutic or a protective immunological (memory) response such that resistance to new infection will be enhanced and/or the clinical severity of the disease reduced. Such protection will be demonstrated by either a reduction in number of symptoms, severity of symptoms, or the lack of one or more of the symptoms associated with the infection of a pathogen, and/or a delay in the of onset of symptoms.

“Immunodeficiency disease causing agent” refers to any and all agents capable of causing an immunodeficiency disease in a subject. Exemplary immunodeficiency disease causing agents include, without limitation, lentiviruses such as HIV, FIV, SIV, CAEV, variants thereof and others known in the art.

“Isolated” means altered “by the hand of man” from its natural state, i.e., if it occurs in nature, it has been changed or removed from its original environment, or both. For example, a polynucleotide or polypeptide naturally present in a living organism is not “isolated,” but the same polynucleotide or polypeptide separated from the coexisting materials of its natural state is “isolated”, as the term is employed herein.

Herein, “pharmaceutical-acceptable carrier” or “veterinary-acceptable carrier” include any and all solvents, dispersion media, coatings, stabilizing agents, growth media, dispersion media, cell culture media and cell culture constituents, coatings, adjuvants, diluents, preservatives, antibacterial and antifungal agents, isotonic agents, adsorption delaying agents, and the like.

As used herein, “subject” refers to a living organism having a central nervous system. In particular, subjects include, but are not limited to, human subjects or patients and companion animals. Exemplary companion animals may include domesticated mammals (e.g., dogs, cats, horses), mammals with significant commercial value (e.g., dairy cows, beef cattle, sporting animals), mammals with significant scientific values (e.g., captive or free specimens of endangered species), or mammals which otherwise have value. Suitable subjects also include: mice, rats, dogs, cats, ungulates such as cattle, swine, sheep, horses, and goats, lagomorphs such as rabbits and hares, other rodents, and primates such as monkeys, chimps, and apes. In some embodiments, subjects may be diagnosed with a fibroblastic condition, may be at risk for a fibroblastic condition, or may be experiencing a fibroblastic condition. Subjects may be of any age including new born, adolescence, adult, middle age, or elderly.

The term “vaccine” as used herein refers to a composition that induces an immune response in the recipient subject of the vaccine. Methods and compositions described herein cover a nucleic acid, such as a DNA plasmid or vaccine that induces humoral responses, cell-mediated responses, or both in the subject as protection against current or future infection. The vaccine can induce protection against infection upon subsequent challenge with an infectious disease causing agent. Protection refers to resistance, including partial resistance, to persistent infection of a subject.

### EXAMPLES

The following examples are simply intended to further illustrate and explain the present invention. The invention, therefore, should not be limited to any of the details in these examples.

### Example 1

### Materials and Methods

The following methods and materials were used in the subsequent examples.

Animals.

Six week old female BALB/c mice were purchased from Harlan Laboratories. Two 3-5 year old Indian rhesus macaques were purchased and housed in the Laboratory Animal Resources of the University of Kansas Medical Center. All animals were used in accordance with the National Institute of Health and the University of Kansas Medical Center Institutional Animal Care and Use Committee guidelines.

Transfection of HEK 293T Cells for Viral Protein Expression Assessment.

Transfections were performed using a cationic polymer polyethylenamine, ExGen™ 500, according to the protocols provided by the manufacturer (Fermentas, Hanover, Md.) for adherent cells. Supernatant fluids were harvested from HEK-293 T transfected cells 14 hours (h) and 24 h after transfection and assessed for p24 content. Transfected cells were then labeled with 100 μCi of ³⁵S-methionine at 48 h post-transfection and used for immunoprecipitation of viral proteins from the cell lysate and supernatant compartments using a hyperimmune macaque serum that has antibodies against all the viral proteins.

Quantification of Gag p24 Release in the Culture Medium of Transfected Cells.

Gag p24 was assessed by the highly sensitive capture enzyme-linked immunosorbent assay (ELISA) kit (Coulter laboratories, Hialeah, Fla.). A standard curve was prepared for each assay, as per the manufacturer's instructions. The concentrations of Gag p24 were determined from the OD₄₅₀ plotted against a standard curve by linear regression analysis.

Inoculation of Mice and Macaques.

Endotoxin-free vaccine DNA was produced using a BIOFLO 110 modular Fermentor (New Brunswick Scientific, Edison, N.J.) that routinely produce high yield of DNA following plasmid DNA extraction using the standard methods with Qiagen Giga kit.

Two groups of BALB/c mice were inoculated intramuscularly (IM) with a single dose of 200 μg of CAL-Δ4-SHIV_{KU2} and Δ4-SHIV_{KU2} DNA vaccine, respectively. Each mouse was injected with a total of 100 μl of DNA solution prepared in phosphate buffer saline (PBS) at 2 μg/μl DNA; 50 μl in each gastrocnemius muscle.

Macaques were inoculated IM with a single dose of 30 mg of DNA vaccine at 6 mg/ml concentration. All DNAs used to inject the macaques and mice contained at least 90% of the supercoiled form of plasmid. DNA solution was prepared in 5 ml of PBS (0.1 M, pH 7.4) and injected intramuscularly to macaques at ten different sites of the rear legs using a 21 gauge needle.

HIV Peptides.

Overlapping 15-mer peptides, with 11-amino acid overlaps, spanning the entire molecules of HIV Gag, Env, Tat, Rev, and Nef, proteins were obtained from the National Institutes of Health AIDS Research and Reference Reagent Program (catalog nos. 8117, 6451, 5138, 6445, and 5189, respectively). These peptides are based on consensus sequences from Glade B HIV genomes. The HIV DNA vaccine encodes Gag and Nef from the SF2 HIV strain and Tat, Rev, and Env from the HXB2 HIV strain. These two strains are both Glade B viruses.

Processing of Spleen and Blood for Mononuclear Cell Isolations.

Mice were killed at 2 and 4 weeks post-immunization, respectively, and spleen collected. Splenocytes were isolated in Hanks solution, treated with BD lysing solution to remove the crythrocytes, and mononuclear cells were counted.

Peripheral blood samples were collected from vaccinated macaques by venipuncture in sodium heparin coated tubes. PBMCs were isolated from Buffy-coats by centrifugation through Ficoll-Hypaque density gradients. Cells were then used to perform multiparametric flow cytometry assays.

Assays for Detection of HIV-Specific Immune T Cells.

Quantitative ELISPOT assay were performed on splenocytes to measure IFN-γ-producing splenocytes in response to groups of overlapping peptides used at a concentration of 2 μg/ml.

Humanization of Mice with PBMCs.

Nod/SCID mice aged 6 weeks were humanized with human peripheral blood mononuclear cells (PBMCs). All of the blood collected in sodium citrate was centrifuged (2000 g, 10 min, 20° C.) to recuperate the white cell layer between plasma and red blood cells. The cells were diluted three times in PBS/EDTA, gently deposited over a cushion of Ficoll (lymphocyte separation medium) and then centrifuged for 45 minutes at 2000 g at 20° C. The PBMC were recuperated, washed several times in PBS/EDTA and suspended again in PBS X1 at 50×10⁶ PBMC in 0.1 ml and were injected intraperitoneally in each mouse. After 48-72 hours post-humanization, the mice were injected intramuscularly with 50 micrograms of DNA vaccine.

Polychromatic (six-color) flow cytometry analyses were performed on splenocytes and PBMC using a three-laser BD LSRII instrument with standard setting. Data files were collected and analyzed using the FACSDiva software program (version 4.1.2; BD Biosciences, San Jose, Calif.). To monitor the expansion and proliferation of HIV-specific T cells, CFSE-labeled (10⁷ cells/ml in 1 μM CFSE for 10 minutes at 37° C., Molecular Probes, Invitrogen, Carlsbad, Calif.) splenocytes or PBMCs were seeded in 96-deep well tissue culture plates (Nunc, Fisher Scientific, Pittsburgh, Pa.) at a density of 2×10⁶ cells/well in 1 ml of medium alone or loaded with 2 μg/ml of HIV peptides and incubated for 5 days at 37° C. After 5 days of incubation, cells were restimulated for 6 h with medium only or by adding relevant HIV peptides in the presence of 0.5 μg/ml of costimulatory CD28, CD49 mAbs and 10 μg/ml of brefeldin A (Sigma-Aldrich, St. Louis, Mo.). Cells were then washed and stained with anti-CD3, -CD8, -CD4 mAbs for 20 minutes at 4° C. Additionally, eithidium monazide (EMA; Molecular Probes, Invitrogen, Carlsbad, Calif.) was added at 0.5 μg/ml during the surface labeling step to allow exclusion of dead cells in samples that have been cultured for 5 days and restimulated for 6 h. In such a case, all samples were exposed to light for 15 minutes at room temperature to allow EMA to covalently link to the DNA in dead cells prior t permeabilization. Then the cells were fixed/permeabilized (Cytofix/Cytoperm Plus; BD Biosciences, San Jose, Calif.) and stained with anti-IFN-γ and IL-2 mAbs for 30 minutes at room temperature. Cells were washed again (Perm/Wash; BD Biosciences, San Jose, Calif.), fixed in 1% paraformaldehyde in PBS, and stored at 4° C. until flow cytometry analysis. For each experiment, unstained and all single-color controls were processed to allow proper compensation as well as all fluorescence-minus-one controls to determine proper population gates. Each analysis was gated on low forward and side scatter lymphocytes (FSC/SSC), EMA⁻, CD3⁺, and high CD8⁺ population to allow the collection of 25,000-50,000 CD8⁺ events (>106 total events). Data were displayed as two-color or density dot plots to measure the proportion of the single-positive or double-positive cells in the highly CD3⁺CD8⁺ population. Bioexponential display was also used to show each population in its entirety.

### Example 2

### Construction of the CAL-Δ4 DNA Construct

The CAL-Δ4 DNA construct comprises antigens for vpx, vpr, gag, pro, vpu, tat, rev, env, and nef proteins. The sequences encoding vpx and vpr antigens were derived from SIV-mac239, and those encoding gag, pro, vpu, tat, rev, env, nef and a portion of rt are derived from HIV. The expression of these antigens is controlled by the CAEV 5′LTR, which controls transcription independently of tat protein presence. Further, the CAEV 5′LTR sequence lacks integrase recognition sequences, which would allow int of HIV to induce integration of the construct into the host DNA.

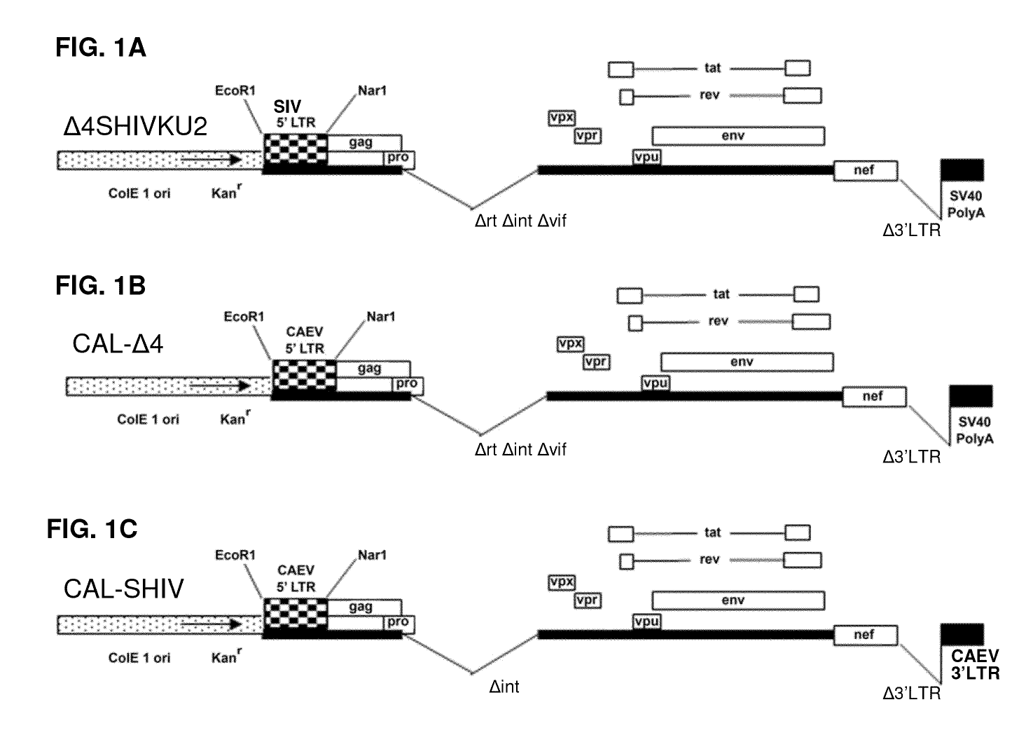

FIG. 1 is a schematic diagram of the CAL-Δ4 DNA construct (SEQNO: 1) of the present invention. The construction of the present DNA vaccine CAL-Δ4 DNA construct (SEQ ID NO: 1) is performed as follows. The vector used for the present vaccine is pET-9a. The 2.3 kb EcoR I/Xmn I fragment of pET-9a is replaced with the approximately 7.4 kb modified SHIVₖᵤ₂ provirus genome and the approximately 0.5 kb polyadenylation signal sequence of SV40 to yield an intermediate vector. EcoRI and Not I restriction sites were created immediately upstream of the 5′ LTR and at the end of the nef gene, respectively, in another intermediate vector. The reverse transcriptase (rt), integrase (int), and vif genes are rendered non-functional by deletion of an approximately 2.5 kb DNA fragment between the downstream end of the pro gene and upstream of the vpx gene. In particular, the pol gene, which encodes rt, int, and vif, is truncated such that 80% of the coding sequence for rt is removed and all of the coding sequence for int and vif is removed as well as that of the 3′LTR. The approximately 3.8 kb nucleotide sequence that encodes the envelope (env), nef, and 3′ LTR genes of SHIVₖᵤ₂ provirus genome is then replaced with the approximately 3.2 kb EcoRV/Not I DNA fragment that encodes the env and nef genes of HIV-1. The approximately 2.5 kb Nar I/BstE II DNA fragment that encodes the leader sequence, gag, and pro genes of SIV_{mac239} in SHIVₖᵤ₂ is replaced with an approximately 2.4 kb Nar I/BstE II fragment that encodes the HIV-1 leader sequence, gag, and pro of HIV-1 to yield Δ4-SHIV1ₖᵤ₂ DNA construct (SEQ ID NO: 8). Thus, the 5′ LTR, vpx, and vpr genes of the Δ4-SHIVₖᵤ₂ DNA vaccine are from SIV_{mac239}, and the gag, pro, tat, rev, vpu, env, and nef are from HIV-1.

Next, 830 bp of the SIV 5′ LTR sequences were removed by following double digestion with EcoRI and NarI enzymes and ligation of the caprine arthritis encephalitis goat lintivirus (CAEV) 5′LTR, obtained by PCR amplification of the 450 bp and double digestion with EcoRI and NarI enzymes. Thus, the vpx, and vpr genes of the CAL Δ4-SHIV DNA vaccine are from SIV_{mac239}, the gag, pro, tat, rev, vpu, env, and nef are from HIV-1, and the 5′ LTR is from CAEV.

Unlike the HIV and SIV 5′LTRs, the CAEV 5′LTR is not dependent on the presence of Tat protein to activate transcription. Dependence on Tat to express proteins limits potential efficacy, since the expression of antigens is dependent on the amount of Tat available. Not all cells may express Tat in an infected subject. Therefore, only cells with Tat will express the antigens of the vaccine. The CAEV 5′LTR is not dependent on Tat and all cells harboring the DNA vaccine construct will express the antigens.

Also, the CAEV 5′LTR does not contain integrase recognition sequences as the SIV 5′LTR does. Absence of integrase recognition sequences, in addition to the lack of a 3′ LTR, prevents integration of the vaccine DNA into the host genome. Also, without the ability of integration into the host genome, any new recombinants that may form will be destroyed due to the inability to integrate and replicate. While the Int protein is not encoded by the CAL-Δ4-SHIV DNA vaccine, vaccinated subjects already infected with an immunodefficency virus have Int protein present. Lack of the integrase recognition sequences enhances the safety of CAL-Δ4-SHIV1 DNA above that of other vaccines known in the art.

Further, use of the CAEV 5′LTR preserves the balance of gene expression and profile of protein expression used by the native LTR. DNA vaccines using constitutive promoters, such as the well-known CMV promoter are not as effective as those using viral LTRs to regulate gene expression. Use of such promoters does not allow antigen expression in the way of the native virus.

The information below is provided to detail the structure of the CAL-Δ4 DNA construct (SEQ ID NO: 1) more completely. A 4,981 bp fragment of SHIVₖᵤ₂ that encodes the entire gag, and pol genes (which therefore includes the rt and int portions of the genome), as well as the first 472 bp of the vif gene, is replaced with a 2,376 bp DNA fragment of HIV-1 in the Δ4-SHIVₖᵤ₂ DNA construct. This 2,376 bp fragment encodes the entire HIV-1 gag gene, and a portion of the HIV-1 pol gene (the entire region encoding protease is included; the nucleotides corresponding to the first 104 amino acids of reverse transcriptase have been removed; the int and vif genes have been completely removed). The 4,981 bp fragment of SHIVₖᵤ₂ that was replaced is designated SEQ ID NO: 5. The DNA sequence of the first 472 bp of the vif gene of SHIVₖᵤ₂, which was also replaced is designated SEQ ID NO: 6. The DNA sequence of the 2,376 bp fragment of HIV-1 used to replace the deleted 4,981 bp and 472 bp SHIVₖᵤ₂ sequences (SEQ ID NO: 5 and SEQ ID NO: 6, respectively) is designated SEQ ID NO: 7.

In addition to the above, a 411 bp DNA fragment is deleted from the 3′ LTR of SHIVₖᵤ₂ to yield the CAL-Δ4 DNA construct (SEQ ID NO: 1). This deleted 3′ LTR sequence is designated SEQ. ID NO: 8. In the CAL-Δ4 DNA construct, the deleted 3′LTR sequences are replaced with 481 bp DNA sequence of the SV40 polyadenylation signal sequence that is designated SEQ ID NO: 4. CAL-Δ4 genome lacks the SIV 5′ LTR, which was replaced with the equivalent sequences from CAEV (SEQ. ID NO: 2).

### Example 3

### Antigen Presentation by the CAL-Δ4 DNA Vaccine

DNA vaccines present antigens by way of expressing proteins encoded in their coding sequence. The coding sequence of the CAL-Δ4 DNA vaccine encodes the following proteins: vpx, vpr, gag, pro, tat, rev, vpu, env, and nef. To determine if these proteins were expressed by the administration of the CAL-Δ4 DNA vaccine, the following assays were conducted.

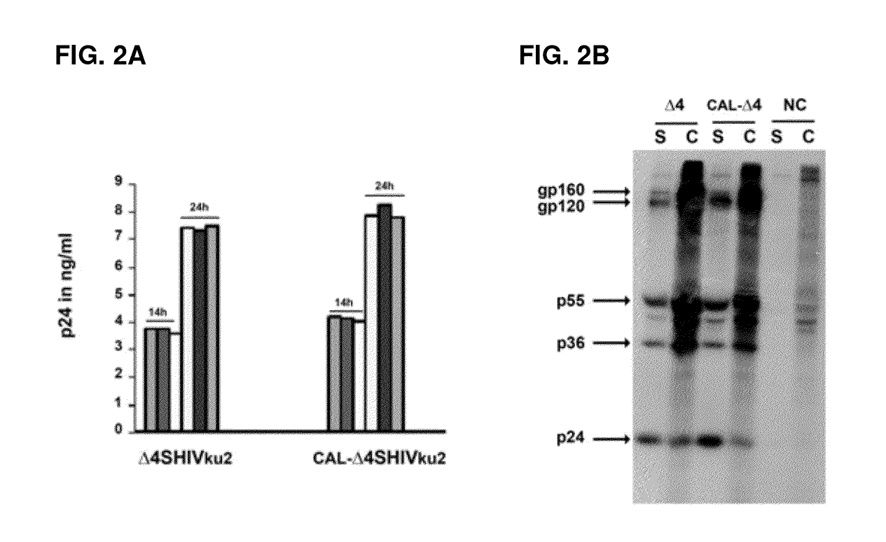

The expression of proteins encoded by the CAL-Δ4 DNA vaccine construct were analyzed using human embryonic kidney 293 cells (HEK 293 T) transfected with either the CAL-Δ4 DNA vaccine of the present invention or Δ4SHIV_{KU2} DNA vaccine. The Δ4SHIV_{KU2} DNA vaccine uses the SIV 5′ LTR rather than the CAEV promoter sequence used in the DNA vaccine of the invention. In order to evaluate the efficacy of protein expression, we first examined by enzyme-linked immunosorbent assay (ELISA) the amount of Gag p24 antigen that was released by HEK-293T transfected cells with each of the two DNA vaccine contructs. Transfections were performed using a cationic polymer polyethylenamine, ExGen™ 500, according to the protocols provided by the manufacturer (Fermentas, Hanover, Md.) for adherent cells. Supernatant fluids were harvested from transfected cells 14 hours (h) and 24 h after transfection and assessed for Gag protein content. Gag protein content was assessed by the highly sensitive capture ELISA kit (Coulter laboratories, Hialeah, Fla.). A standard curve was prepared for each assay, as per the manufacturer's instructions. The concentrations of Gag protein were determined from the OD₄₅₀ plotted against a standard curve by linear regression analysis. Triplicate measurements were performed and the results are representative of two independent experiments (FIG. 2A). As shown in FIG. 2A, both DNA vaccines produced similar amounts of Gag p24 protein secreted at the two time points. In summary, the CAL-Δ4 DNA vaccine construct was capable of expressing the encoded Gag protein similarly to the Δ4SHIV_{KU2} DNA vaccine (FIG. 2A).

Also, viral protein profiles were evaluated in transfected cells by using an anti-SHIV monkey serum for the radioimmunoprecipitation assay of ³⁵S-methionine labeled proteins. After 48 hours, proteins of transfected HEK293T cells were labeled with ³⁵S-methionine. Viral proteins were immunoprecipitated from the cell lysate (C) and supernatant (S) compartments using a hyperimmune macaque serum that contained antibodies against all of the CAL-Δ4 DNA encoded proteins. The proteins expressed by the CAL-Δ4 DNA vaccine construct were detected by radio-immunoprecipitation (FIG. 2B) and sizes of the major proteins are indicated in kDa. As shown in FIG. 2B, no substantial difference of viral protein profiles were detected in the cell lysates and supernatant fluids indicating that Δ4 SHIV_{KU2} and CAL-Δ4 DNA vaccine contructs express HIV viral proteins with similar efficiency.

The CAL-Δ4 DNA construct was capable of expressing the encoded antigenic proteins in human cells.

### Example 4

### Efficacy of the CAL-Δ4 DNA Vaccine

In order to demonstrate the efficacy of the CAL-Δ4 DNA vaccine, the following T-cell response experiment was conducted. The following study shows that the CAL-Δ4 DNA vaccine modifies the vaccine-induced T cell response to all expressed HIV antigens in injected mice.

Six-week-old BALB/c mice were inoculated intramuscularly with a single dose of 200 μg of CAL-Δ4 DNA or Δ4SHIV_{KU2} DNA vaccine prepared in phosphate buffer saline (PBS) at 2 μg/ul DNA. Each mouse was injected with a total of 100 μl of DNA solution, 50 μl in each gastrocnemius muscle. Mice were killed at 2 and 4 weeks post-immunization, respectively, and spleens were then collected. Splenocytes were collected in Hanks solution, treated with BD lysing solution to remove the erythrocytes, and then mononuclear cells were counted.

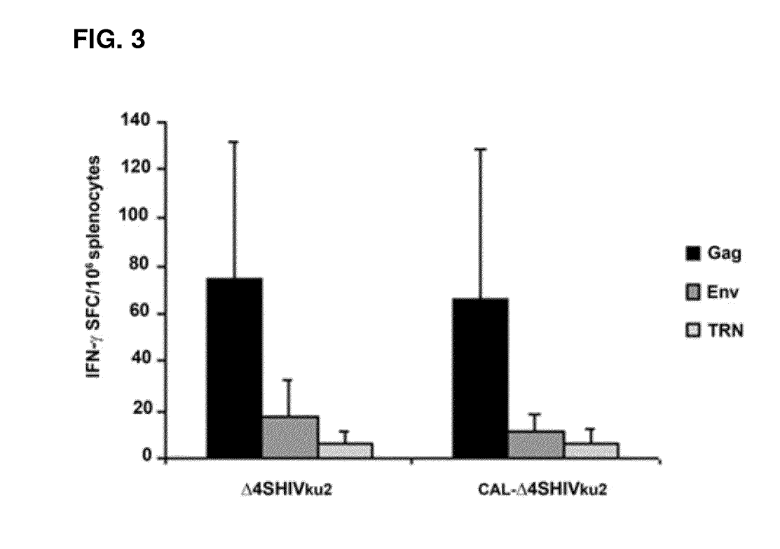

Quantitative ELISPOT assay were performed on splenocytes to measure IFN-γ-producing splenocytes in response to groups of overlapping peptides used at a concentration of 2 μg/ml. IFN-γ ELISPOT responses within 2 to 4 weeks post-immunization were evaluated using pools of Gag, Env, TR (Tat+Rev and Nef combined) HIV peptides. As shown in FIG. 3, at 2 weeks post-inoculation, mice immunized with CAL-Δ4SHIV vaccine developed IFN-γ secreting splenocyte responses against Gag, Env, and TRN, similar to those induced by Δ4SHIV_{KU2} DNA vaccine injected mice. The data show the mean of the measurements and the standard deviation (represented by the error bars) obtained using 5 immunized animals in each group (FIG. 3, SFC: spot-forming cells).

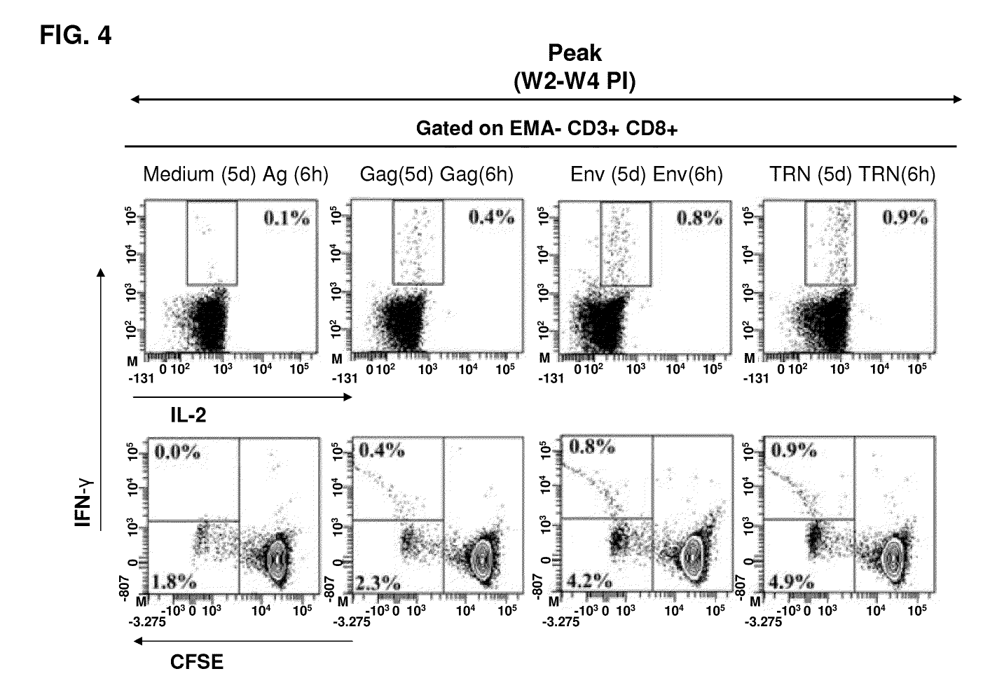

To further examine the profile of the vaccine-induced T cell responses, multiparametric flow cytometry was used to analyze antigen-specific proliferation (CFSE dilution) and IFN-γ secretion of splenocytes in response to HIV antigens. As shown in FIG. 4, 0.3% to 0.8% of cells produced IFN-γ and 0.9% to 4% underwent proliferation with Gag, Env and TRN peptides. The vast majority of the proliferating T cells did not produce detectable IFN-γ upon restimulation. Thus, immunization of mice with CAL-Δ4 induced HIV-specific CD8+ T cells responses that qualitatively and quantitatively resembled the responses induced by the original Δ4SHIV_{KU2} DNA vaccine when injected in mice.

### Example 5

### HIV Specific T Cell Response in Macaques Immunized with Cal-Δ4 DNA Vaccine

In order to demonstrate the efficacy of the CAL-Δ4 DNA vaccine, the following T-cell response experiment was conducted. The following study shows that the CAL-Δ4 DNA vaccine modifies the vaccine-induced T cell response to all expressed HIV antigens in injected macaques.

Macaques were inoculated intramuscularly with a single dose of 30 mg of DNA vaccine at 6 mg/ml concentration. Endotoxin-free vaccine DNA was produced using a BIOFLO 110 modular Fermentor (New Brunswick Scientific) that routinely produces high yield of DNA following plasmid DNA extraction using the standard methods with Qiagen Giga kit. All DNAs used to inject the macaques and mice contained at least 90% of the supercoiled (ccc) form of the plasmid. DNA solution was prepared in 5 ml of PBS (0.1 M (pH 7.4)) and injected intramuscularly at ten different sites of the rear legs using a 21 gauge needle. Peripheral blood was collected in vaccinated macaques by venipuncture in sodium heparin coated tubes and was centrifuged to separate plasma and blood cells. Plasma was frozen and used for detection of antibodies against HIV proteins. PBMCs were isolated from Buffy-coats by centrifugation through Ficoll-Hypaque density gradients. Cells were then divided in 2 fractions and used to perform ELISPOT assays and/or multiparametric flow cytometry assays.

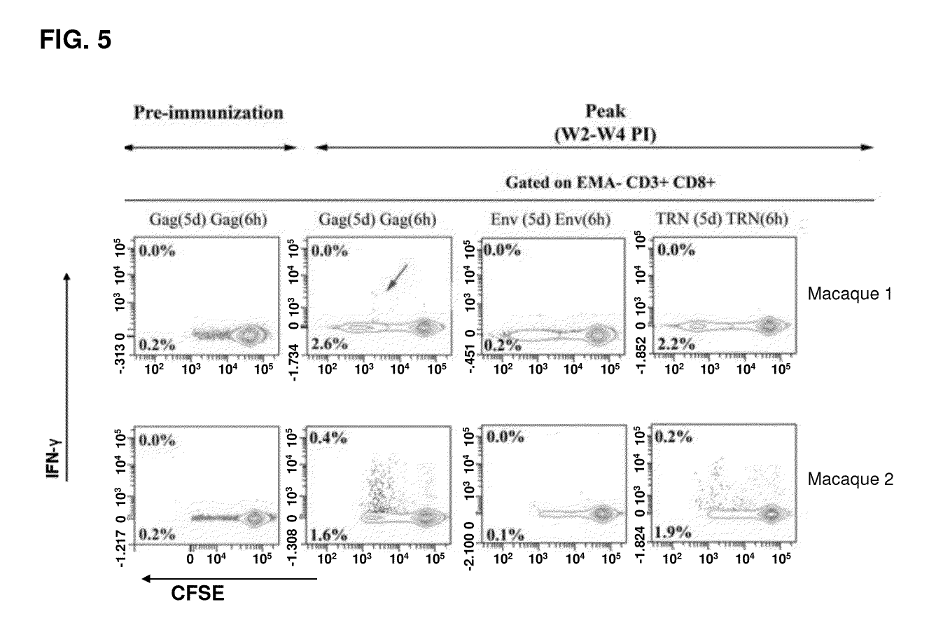

Two rhesus macaques were immunized with a single dose of 30 mg of CAL-Δ4SHIV. At the indicated pre-immunization and post-immunization times, PBMCs were collected, labeled with CFSE, cultured, restimulated and stained using the same procedure as described in Example 4. A multiparametric flow cytometry based assay, showed in both animals that 1.7% to 2% of total CD3+ CD8+ T cells proliferated, but only 0.2% produced IFN-γ in response to TRN mix of peptides (FIG. 5). A similar response was measured against Gag showing that 1.4% to 2.4% of the CD3+ CD8+ T cells proliferated, while only 0.4% produced IFN-γ. No response against Env was measured at these early time-points.

### Example 6

### Efficacy of the CAL-SHIV DNA Vaccine

In order to demonstrate the efficacy of the CAL-SHIV DNA vaccine, the following T-cell response experiment was conducted. The following study shows that the CAL-SHIV DNA vaccine modifies the vaccine-induced T cell response to all expressed HIV antigens in injected mice.

Six-week-old BALB/c mice were inoculated intramuscularly with a single dose of 200 μg of CAL-SHIV DNA, Δ4SHIV_{KU2} or SHIV_{KU2} DNA vaccine prepared in phosphate buffer saline (PBS) at 2 μg/ul DNA. Each mouse was injected with a total of 100 μl of DNA solution, 50 μl in each gastrocnemius muscle. Mice were killed at 2 and 4 weeks post-immunization, respectively, and spleens were then collected. Splenocytes were collected in Hanks solution, treated with BD lysing solution to remove the erythrocytes, and then mononuclear cells were counted.

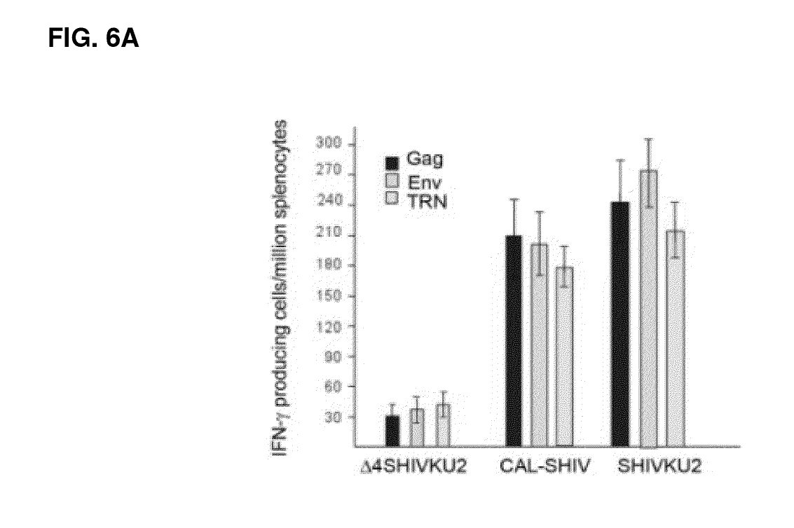

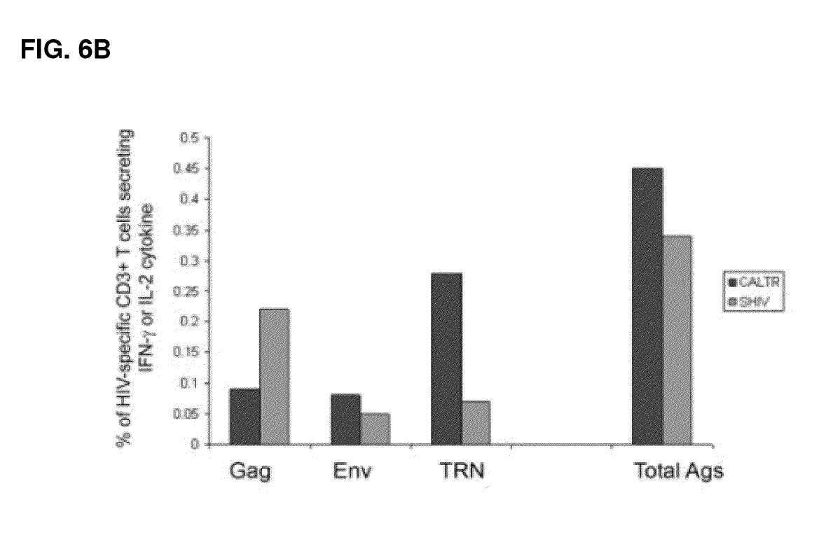

Quantitative ELISPOT assay were performed on splenocytes to measure IFN-γ-producing splenocytes in response to groups of overlapping peptides used at a concentration of 2 μg/ml. IFN-γ ELISPOT responses within 2 to 4 weeks post-immunization were evaluated using pools of Gag, Env, TRN (Tat+Rev and Nef combined) HIV peptides. As shown in FIG. 6A, at 2 weeks post-inoculation, mice immunized with CAL-Δ4SHIV vaccine developed IFN-γ secreting splenocyte responses against Gag, Env, and TRN, similar to those induced by SHIV_{KU2} DNA vaccine injected mice. The data show the mean of the measurements and the standard deviation (represented by the error bars) obtained using 5 immunized animals in each group. The percentage of HIV-specific CD3+ T cells secreting IFN-γ or IL-2 cytokine is depicted in FIG. 6B.

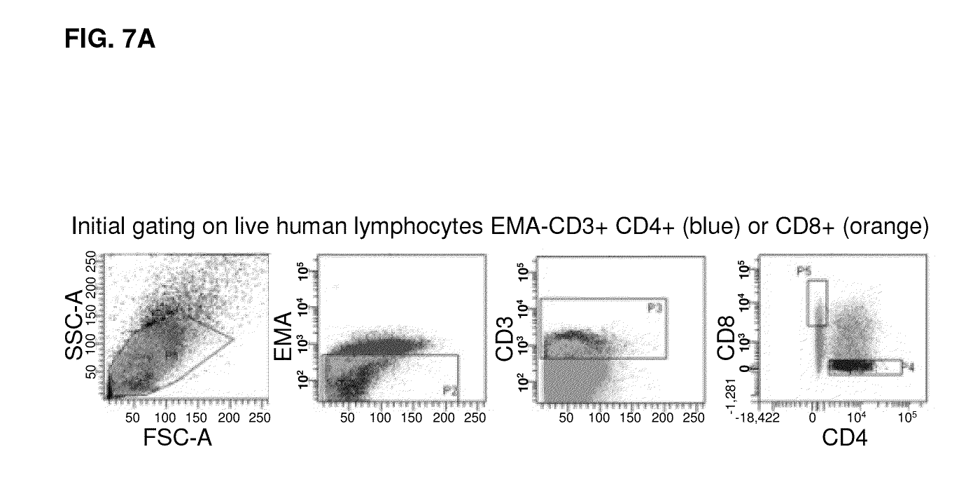

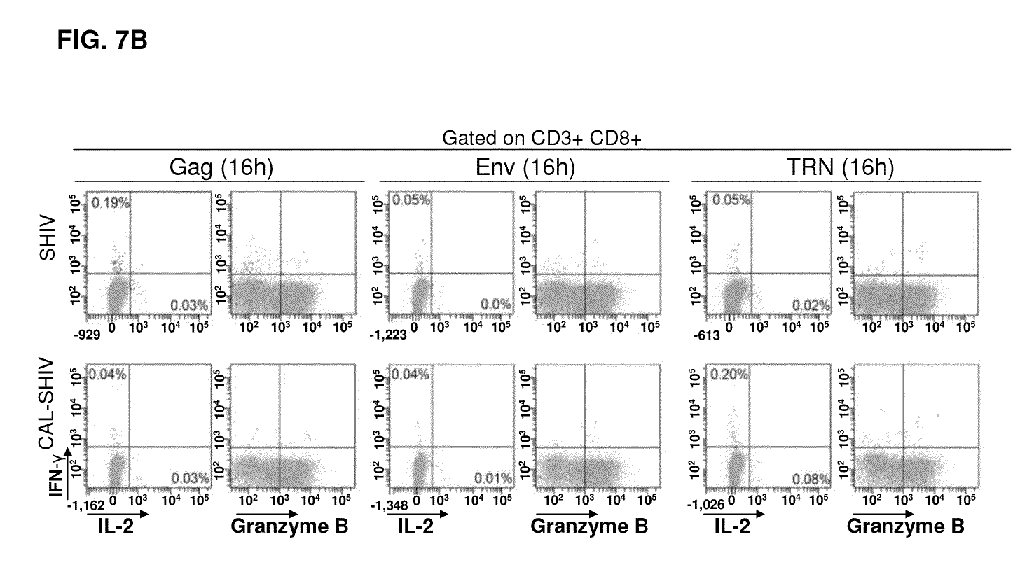

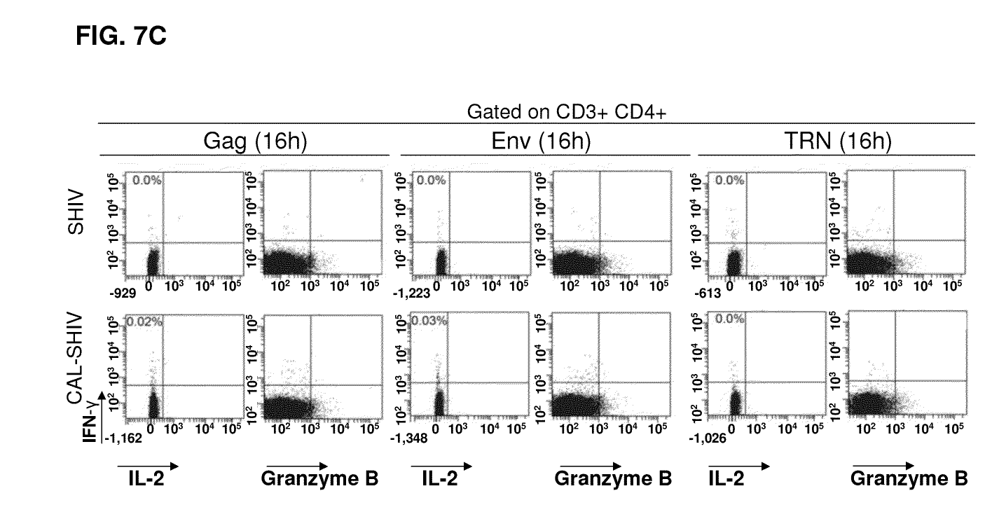

To further examine the profile of the vaccine-induced T cell responses, multiparametric flow cytometry was used to analyze the presence of pathogen specific T cells (IFN-γ, Granzyme B and IL-2 detection) of splenocytes in response to HIV antigens (FIG. 7). As shown in FIG. 7B, the CD8+ cells secreted granzyme B and IL-2 in response to antigen stimulation, and secreted little IFN-γ. Likewise, CD4+ T cells secreted Granzyme B and IL-2 and little IFN-γ (FIG. 7C).

### Example 7

### Efficacy of CAL-SHIV DNA Vaccine on Human PBMCs

In order to demonstrate the efficacy of the CAL-SHIV DNA vaccine in relation to human immunity, the following T-cell response experiment was conducted. The following study shows that the CAL-SHIV DNA vaccine modifies the vaccine-induced T cell response of human PMBCs to all expressed HIV antigens.

Immune deficient NOD/SCID β2 mice were humanized with human PBMCs and then immunized with 50 μg of CAL-SHIV DNA DNA vaccine. Immune cells were isolated from mice over the course of 20 weeks following immunization (PI).

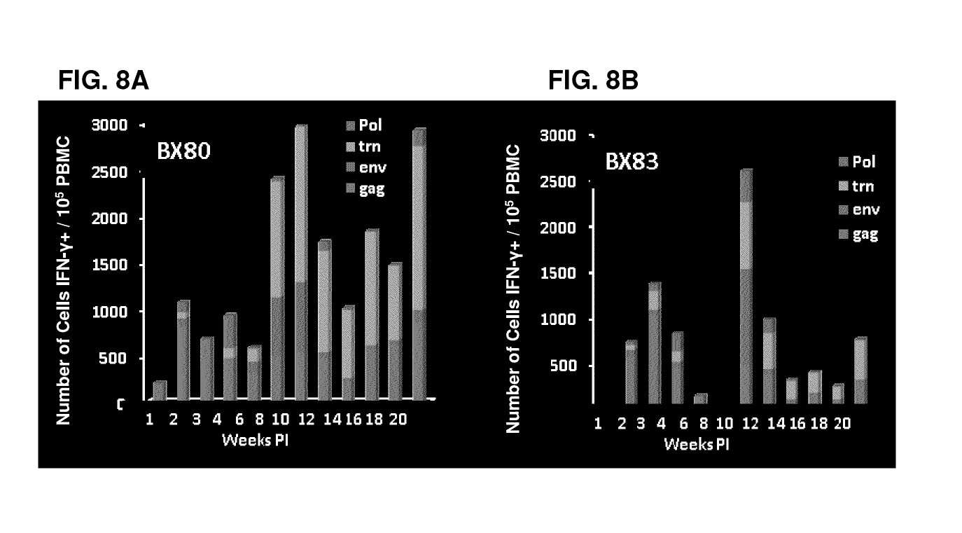

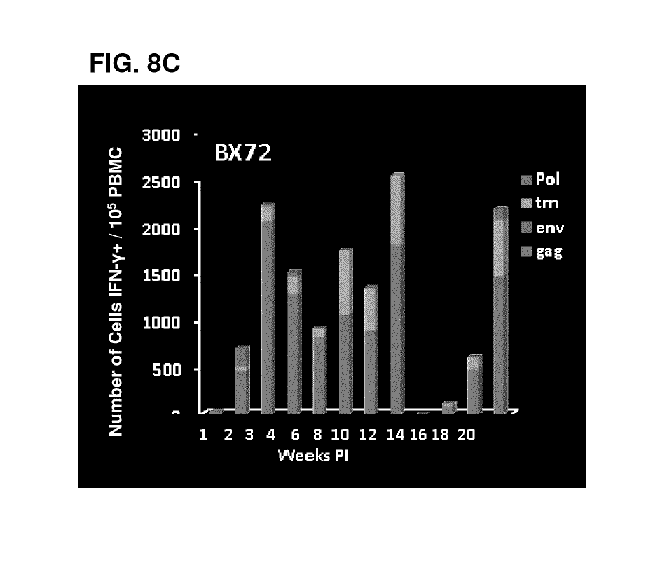

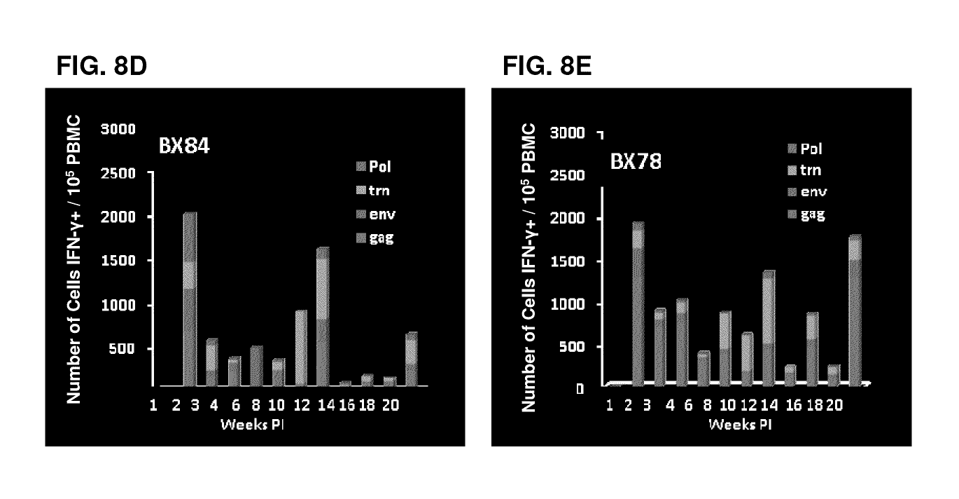

Quantitative ELISPOT assay were performed to measure IFN-γ-producing cells in response to groups of overlapping peptides used at a concentration of 2 μg/ml. IFN-γ ELISPOT responses were evaluated using pools of Gag, Env, Pol, and TRN (Tat+Rev and Nef combined) HIV peptides. As shown in FIG. 8A-E, mice immunized with CAL-SHIV vaccine developed IFN-γ secreting cell responses against Gag, Env, Pol, and TRN.

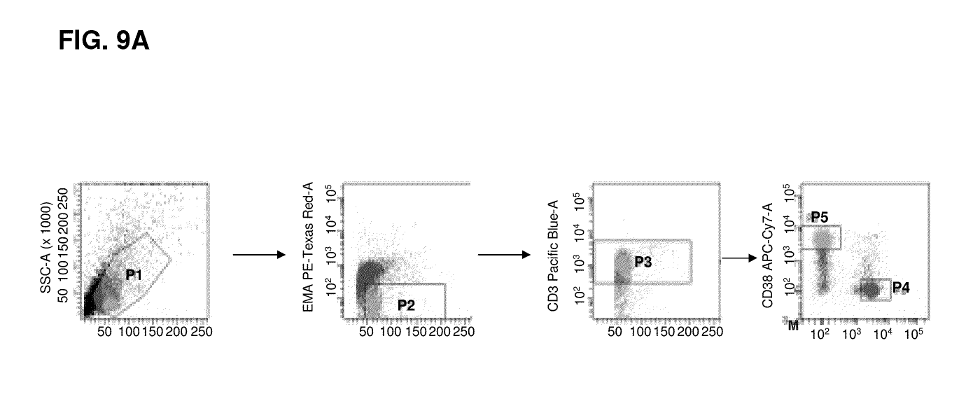

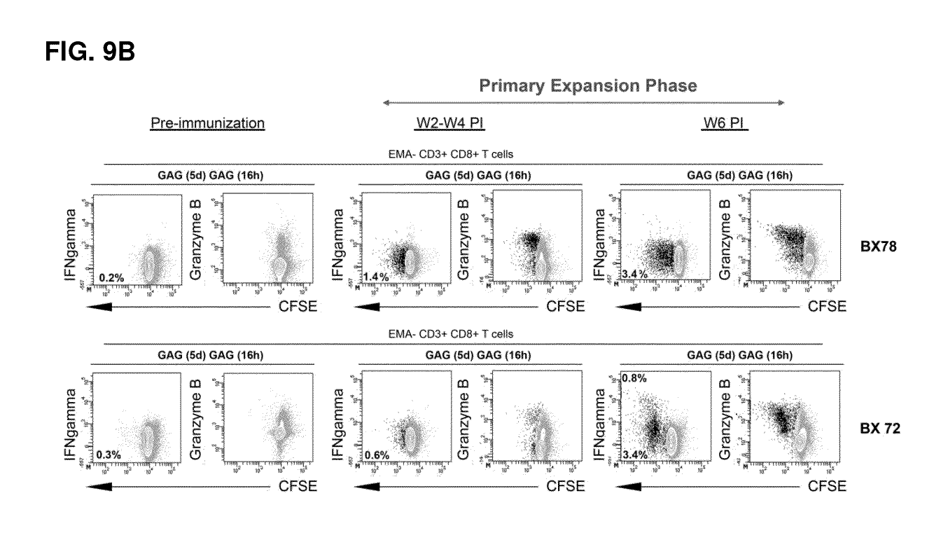

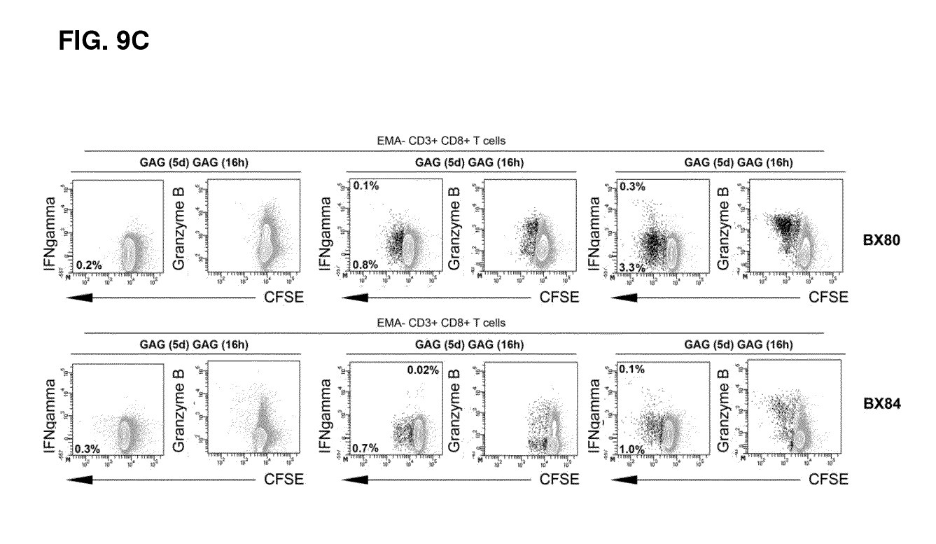

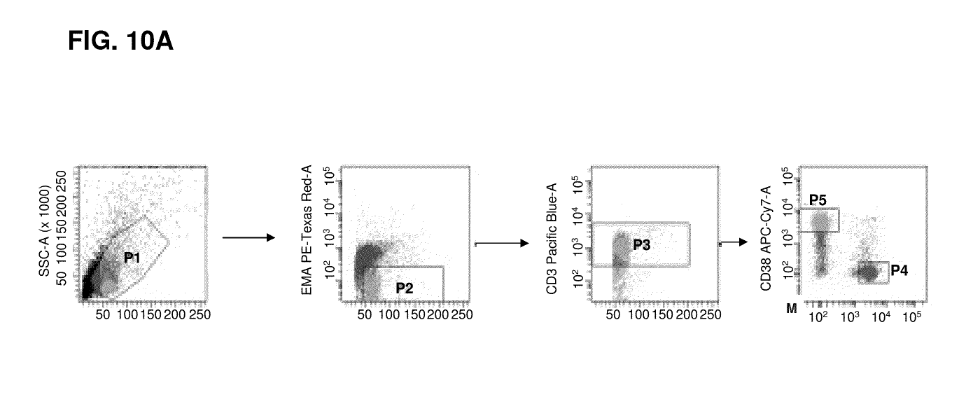

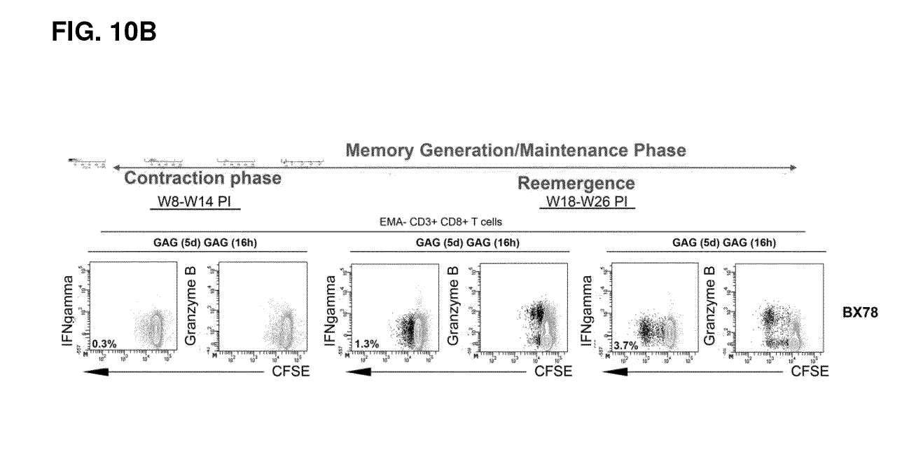

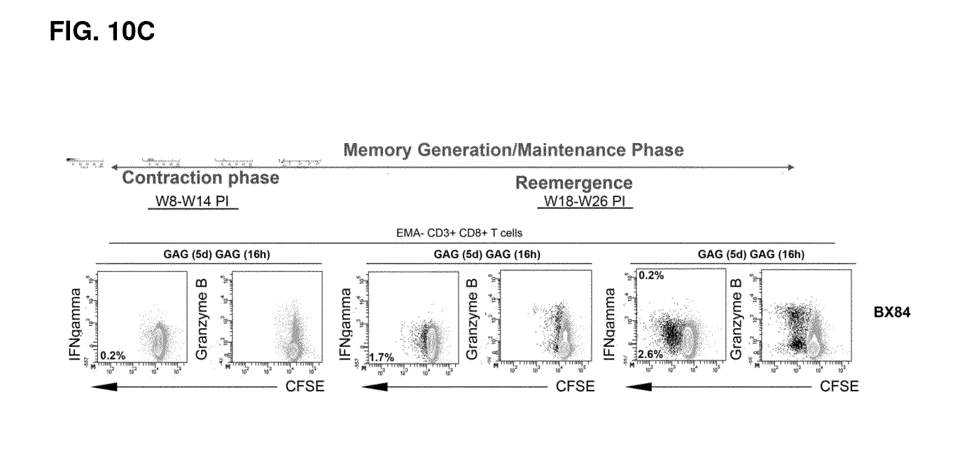

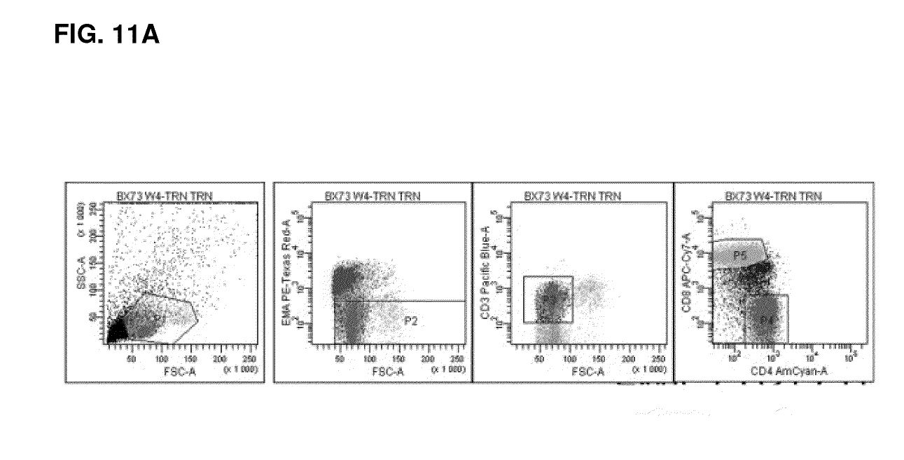

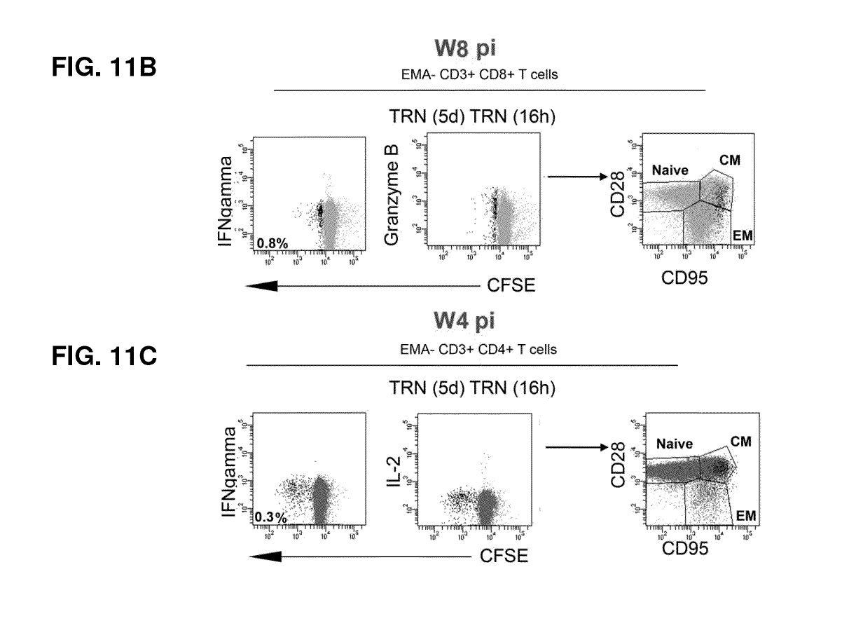

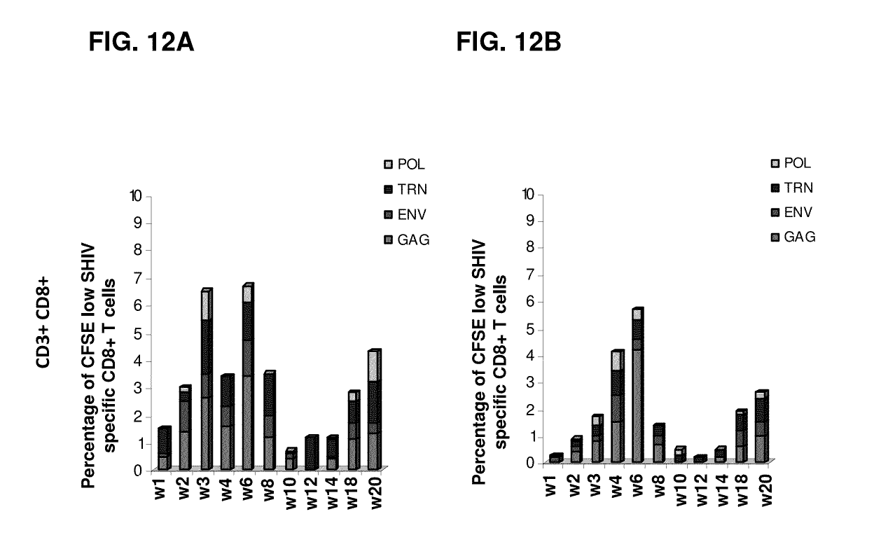

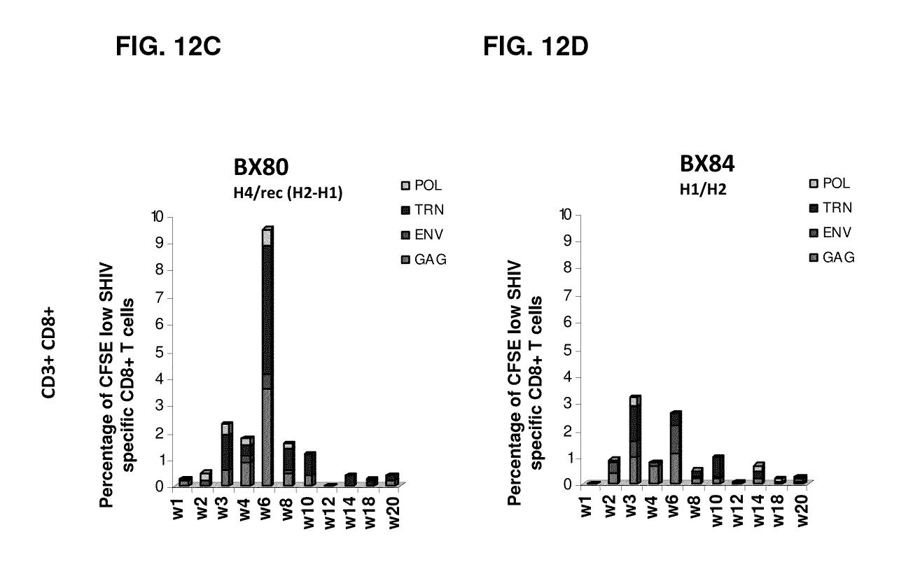

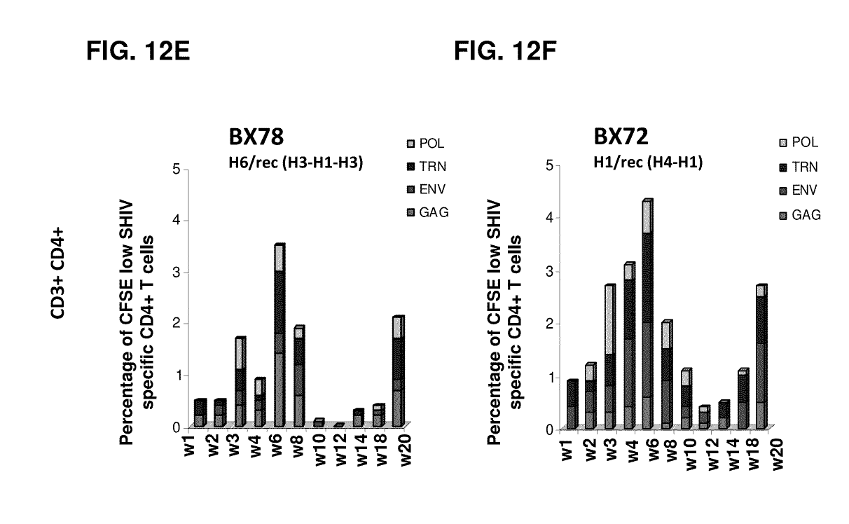

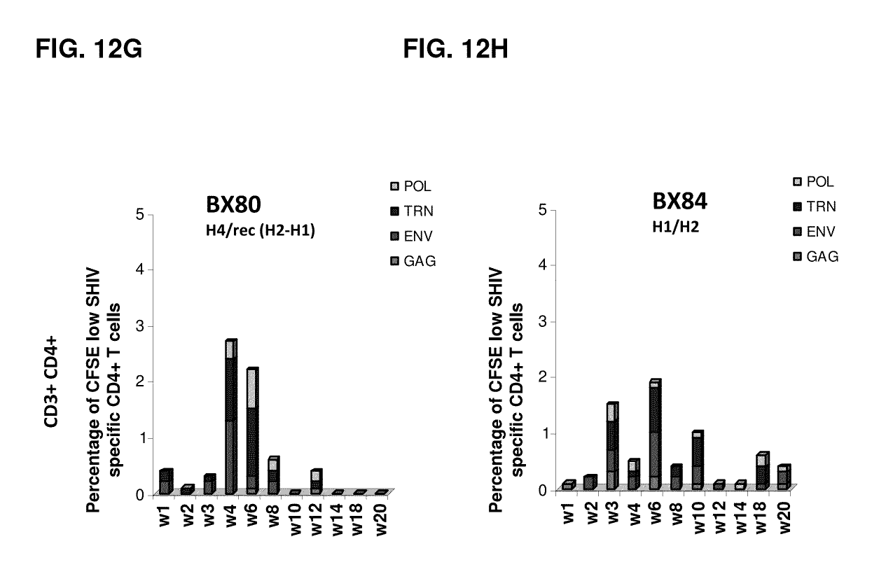

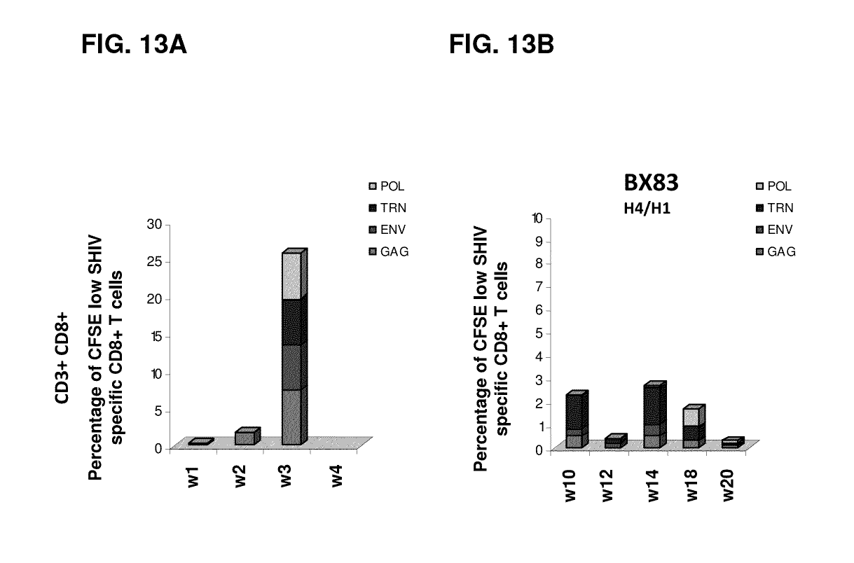

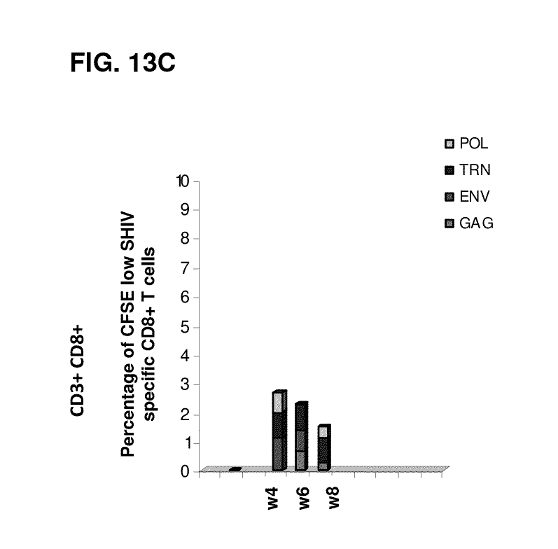

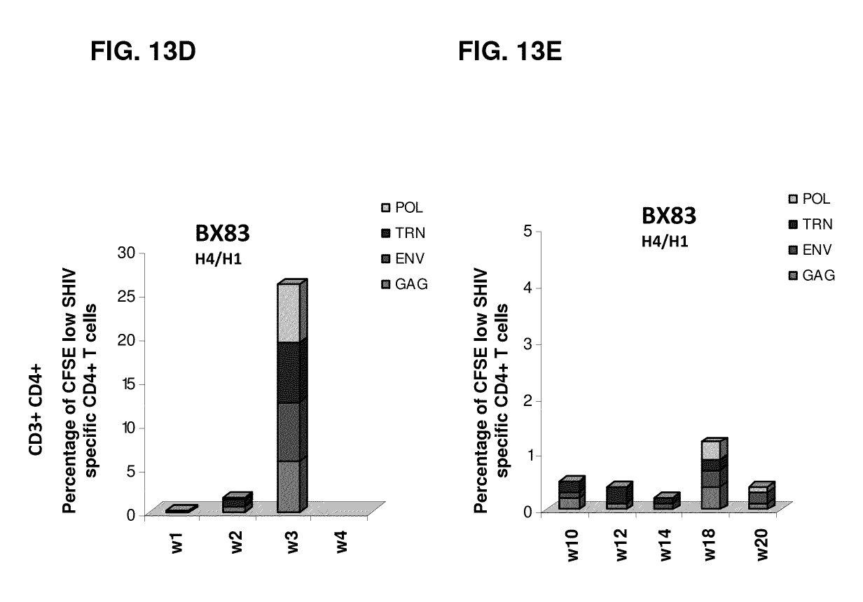

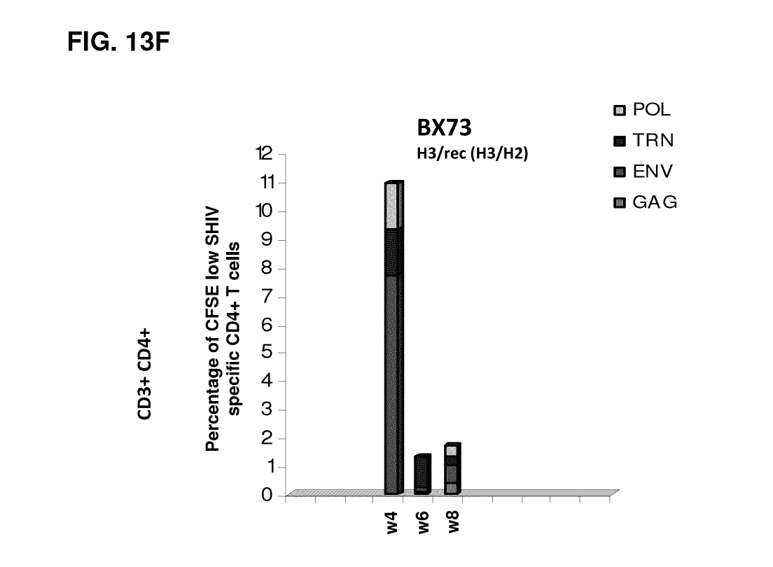

To further examine the profile of the vaccine-induced T cell responses, multiparametric flow cytometry was used to analyze the T cell development phase before and after immunization with CAL-SHIV. The presence of pathogen specific T cells (IFN-γ and Granzyme B detection) increased during the primary expansion phase weeks 2-6 after immunization (FIG. 9A-C). Pathogen specific T cell proliferation subsided during the contraction phase of weeks 8-14 after immunization (FIG. 10A-C). Then the T cell proliferation increased again during the reemergence phase of weeks 18-26 after immunization (FIG. 10A-C). During weeks 4 and 8 the phenotype of the proliferating T cells was analyzed. T cells identified by TRN peptides were identified as Naïve, central memory, and effector T cells (FIG. 11A-C). FIGS. 12 and 13 show the percentage of CD8+ and CD4+ cells recognizing antigens encoded by the CAL-SHIV DNA vaccine for individual humanized mice (FIG. 12A-H and 13A-F).

The data show that the immune responses are directed against all expressed antigens of the DNA vaccine. In absence of any boost with CAL-SHIV there is a second phase of expansion of pathogen specific T cells.

The invention illustratively disclosed herein suitably may be practiced in the absence of any element, which is not specifically disclosed herein. It is apparent to those skilled in the art, however, that many changes, variations, modifications, other uses, and applications to the method are possible, and also changes, variations, modifications, other uses, and applications which do not depart from the spirit and scope of the invention are deemed to be covered by the invention, which is limited only by the claims which follow.

What is claimed is:

1. A DNA composition comprising:
a. a first nucleotide sequence encoding at least one regulatory sequence derived from CAEV sequence; and
b. a second nucleotide sequence encoding at least one immunogenic molecule capable of stimulating an immune response in a subject.

2. The DNA composition of claim 1, wherein the immunogenic molecule is capable of stimulating an immune response against infectious disease causing agents for diseases selected from the group consisting of HIV, SIV, FIV, and combinations thereof.

3. The DNA composition of claim 1, wherein the second nucleotide sequence encodes at least one protein selected from the group consisting of rt, int, vif, gag, pro, vpx, vpr, vpu, nef, tat, env, rev, a 3′ LTR, and combinations thereof.

4. The DNA composition of claim 3, wherein the second nucleotide sequence encodes a non-functional protein selected from the group consisting of rt, int, vif, and combinations thereof.

5. The DNA composition of claim 1 further comprising a third nucleotide sequence encoding a termination sequence selected from the group consisting of a 3′LTR or an SV40 polyadenylation sequence.

6. The DNA composition of claim 1, wherein the regulatory sequence is homologus to SEQ ID NO: 2, having a homology selected from the group consisting of 70%, 75%, 80%, 85%, 90%, 95% and 100%.

7. The DNA composition of claim 1, wherein the first and second nucleotide sequences comprise a nucleotide sequence that is homologus to SEQ ID NO: 1, having a homology selected from the group consisting of 70%, 75%, 80%, 85%, 90%, 95% and 100%.

8. A method of stimulating an immune response in a subject comprising administering to the subject a DNA composition, wherein the DNA composition comprises a first nucleotide sequence encoding a regulatory sequence derived from CAEV sequence and a second nucleotide sequence encoding at least one immunogenic molecule.

9. The method of claim 8, wherein the DNA composition is in a pharmaceutically acceptable carrier.

10. The method of claim 8 further comprising administering anti-retroviral drug therapy.

11. The DNA composition of claim 8, wherein the immunogenic molecule is capable of stimulating an immune response against infectious disease causing agents for diseases selected from the group consisting of Hepatitis, Herpes, HIV, SIV, FIV, and combinations thereof.

12. The method of claim 8, wherein the nucleotide sequence encodes at least one protein selected from the group consisting of rt, int, vif, gag, pro, vpx, vpr, vpu, nef, tat, env, rev, a 3′ LTR, and combinations thereof.

13. The method of claim 12, wherein the nucleotide sequence encodes a non-functional protein selected from the group consisting of rt, int, vif, and combinations thereof.

14. The method of claim 8, wherein the regulatory sequence is homologus to SEQ ID NO: 2, having a homology selected from the group consisting of 70%, 75%, 80%, 85%, 90%, 95% and 100%.

15. The method of claim 8, wherein the first and second nucleotide sequences comprise a nucleotide sequence that is homologus to SEQ ID NO: 1, having a homology selected from the group consisting of 70%, 75%, 80%, 85%, 90%, 95% and 100%.

16. A vaccine for immunization against an infectious disease comprising an isolated DNA molecule encoding at least one immunogenic molecule capable of stimulating an immune response against the infectious disease and a regulatory sequence derived from CAEV sequence.

17. The vaccine of claim 16, wherein the immunogenic molecule is capable of stimulating an immune response against infectious disease causing agents selected from the group consisting of Hepatitis, Herpes, HIV, SIV, FIV, and combinations thereof.

18. The vaccine of claim 16, wherein the DNA molecule encodes at least one protein selected from the group consisting of rt, int, vif, gag, proo, vpx, vpr, vpu, nef, tat, env, rev, a 3′ LTR, and combinations thereof.

19. The vaccine of claim 16, wherein the DNA molecule is homologus to SEQ ID NO: 1, having a homology selected from the group consisting of 70%, 75%, 80%, 85%, 90%, 95% and 100%.


SEQUENCE LISTING (14 sequences)

SEQ ID NO: 1 - DNA, 9569 bp, Artificial Sequence
  CALTR-Delta4
gaattcgtcaactgtgagacatgggctaaagaggactaataacaagctaggccaaattcctgtaaatcacttggggggttataagaaaagcaagttcactatgacaaagcaaaatgtaaaggccaaattcctgtaaatcacttggggggttataagaaaagcaagttcactatgacaaagcaaaatgtaaccgcaagtgctgacagatgtaacagctgacatatcagctgatgcttgctcatgctgacactgtagctctgagctgtatataaggagaagcttgctgcttgcacttcagagttctaggagagtccctcctagtctctcctctccgaggaggtaccgagacctcaaaataaaggagtgattgccttactgccgagtggagagtgattactgagcggcgcccgaacagggacgcgaaagcgaaagtagaaccagaggagctctctcgacgcaggactcggcttgctgaagcgcgcacggcaagaggcgaggggcggcgactggtgagtacgccatttttgactagcggaggctagaaggagagagatgggtgcgagagcgtcaatattaagcgggggacaattagatagatgggaaaaaattcggttacggccagggggaaagaaaagatataagttaaaacatatagtatgggcaagcagagagctagaacgattcgcagttaaccctggcctgttagaaacagcagaaggctgtagacaaatactgggacagctacaaccatcccttcagacaggatcagaggaacttaaatcattatttaatacaatagctaccctctattgtgtacatcaaagaatagagataaaagacaccaaggaagctttagataagatagaggaagagcaaaacaaaagtaagaaaaaagcacagcaagcagcagctgacacaggaaacagcagcagccaagtcagccaaaattaccctatagtgcagaacgctcagggacaaatggtacatcaggccatatcacctagaactttaaatgcatgggtaaaagtagtagaagaaaaggcttttaacccagaagtaatacccatgtttgcagcattgtcagaaggagccaccccacaagatttaaacaccatgctaaacacagtggggggacatcaagcagccatgcaaatattaaaagagactatcaatgaggaagctgcagaatgggatagattgcatccagtacatgcagggcctattgcaccaggccaaatgagagaaccaaggggaagtgacatagcaggaactactagtacccttcaggaacaaataggatggatgacaaataatccacctatcccagtaggagaaatctataaaaaatggataatcatgggattaaataaaattgtaaggatgtatagccctaccagtattctggacataagacaaggaccaaaggaaccctttagagactatgtagaccggttctataaaactctaagagccgagcaagcttcacaggaagtaaaaaattggatgacagaaaccttgttggtccaaaattcaaaccccgattgtaagactattttaaaagcattaggaccaggagctacactagaagaaatgatgacagcatgccagggagtgggaggacctggccataaagcaagagttttggcagaagcaatgagccaagtaacaaatccaacggccgtgatgatgcagaaaagcaattttaggggccaaagaaaaattgttaagtgttttaattgtggcaaagaagggcacatagccaaaaattgcagggctcctagaaaaaagggctgttggaaatgtggaaaggaaggacaccaaatgaaagattgtactgaaagacaggctaattttttagggaagatctggccttcctacaagggaaggccagggaattttcctcaaagcaggctagaaccaacagccccaccagaagcgagcttcaggtttggggaggagacaacaactccccctcagaagcaggagacgatagacaaggaggtgtatcctttaacctccctcagatcactctttggcaacgacccctcgtcacaataaagataggggggcaactaaaagaagctctattagatacaggagcagatgatacagtgttagaagacatgaatttgccaggaaaatggaaaccaaaaatgatagggggaattggaggatttatcaaagtaaaacagtatgatcagatacccatagaaatctgtggacataaaactataggtacagtattaataggacctacacctgtcaacataattggaaggaatttgttgactcagcttggttgcactttaaattttcccattagtcctattgaaactgtaccagtaaaattaaagccaggaatggatggcccaaaagttaagcaatggccattgacagaagaaaaaataaaagcattaatggagatatgcacagaaatggaaaaggaagggaaaatttcaaaaattgggcctgaaaatccatacaatactccagtgtttgccataaagaaaaaagacagtactaagtggagaaaattagtagatttcagagaacttaataagaaaactcaagacttctgggaggttcaattaggaataccacatcccgcggggttaaaaaagaaaaagtcagtaacagtactggatgtgggtgatgcatacttctcagttcccttagatgaagattttaggaagtatactgcatttaccatacctagtataaacaatgagacatcaggaattagatatcagtacaatgtgcttccacagggatggaaagggtcaccatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttttggaagaactgaaagaagaagctttaaaacattttgatcctcgcttgctaactgcccttggtaatcatatctataatcgtcacggagacactctagagggagcaggagaactcattagaatcctccaacgagcgctcttcatgcatttcagaggcggatgcatccactccagaatcggccaacctgagggaggaaatcctctctcagctataccgccctctagaagcattctgtagagcaagaaatggagccagtagatcctagactagagccctggaagcatccaggaagtaagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagagatatcagaattatcagcacttgtggagagagggcatcttgctccttgggatattaatgatatgtagcactgcaggacaattgtgggtcacagtctattatggggtacctgtgtggagagaagcaaccaccactctattttgtgcatcagatgctaaagcatatgatacagaggtgcataatgtctgggccacacatgcctgtgtacccacagaccccagcccacaagaaatggcattggaaaatgtgacagaaaattttgacatgtggaaaaataatatggtagaacagatgcatgaagatataatcagcttatgggatcaaagcctaaagccttgtgtaaaattaactccactatgtgttactttaaattgcactgatgtaaagagaaatgctactagtaacactagtagtagctgggaaaggatggaaccaggagaaataaaaaactgctctttcaatgtcacctcaaatataagagataagatgcggaaagaatatgcactcttttataaacttgatgtaataccaataaataatactagtgataatagtgctaaatatagattgataagttgtaacacctcagtccttacacaagcttgtccaaaaatatcctttgagccaattccaatacattattgtaccccggctggttttgcgcttctgaagtgtaatgataaggagttcaatggaacgggaccatgtaaaaatgtcagcacagtacaatgtacacatggaatcaagccagtagtatcaactcaactgctgttaaatggcagtctatcagaaggaggggttgtaattagatctcaaaatttcacaaacaatgctaaaaccataatagtacagctgaatgaaactgtagaaattaattgtacaaggcccaacaacaatacaagaagaagtataaatataggaccagggagagcattttatgcagcagaacaaataataggagatataagacaagcacattgtaacattagtagagcaaaatggaataacactttaaaactgatagttggaaaattacaagaacaatttgggaagaaaacaataatctttaatcaatcctcaggaggagaccctgagattgtaacacacagttttaattgtggaggggaatttttctactgtgattcaacacaactgtttaacagtacttggacgaatgaaaataacgggtccaacactaaagggaatgacacaatcatactaccatgcagaataaaacaaattgtaaacctgtggcaggaagtaggaaaagcaatgtatgcccctcccatcagaagaccaattagatgctcatcaaatattacagggctgctactaacaagagatggtggtcctaataggacgaacgagacattcagacctggaggaggagatatgagggacaattggagaagtgaattatacaaatataaagtagtaaaaattgaaccattaggagtagcacccaccaaggcaaagagaagagtggtgcaaagagaaaaaagagcagtgggaataggagctctgttccttgggttcttgggaacagcaggaagcactatgggcgcagcgtcactgacgctgacggtacaggccagacaattattgtctggtatagtgcaacagcagaacaatttgctgagagctattgaagcgcaacaacatctgttgcagctcacagtctggggcatcaagcagctccaggcaagagtcctggctgtggaaagatacctaagggatcaacagctcctgggaatttggggttgctctggaaaactcatttgcaccactgctgtgccttggaacactagttggagtaataaatctctagatgacatttggaacaacatgacttggatgcagtgggaaagagaaattgacaattacacaaacacaatatacaccttacttcaggaatcacaactccaacaagaacagaatgaaaaagaactattggaattggataaatgggcaagtttgtggaattggttcgatataacaagttggctgtggtatataaaaatattcataatgatagtaggaggcttgataggtttaagaatagtttttactgtactttctatagtgaatagagttaggaagggatactcaccattatcgttccagacccaccgcccagctccagggggacccgacaggcccgaaggaatcgaagaagaaggtggagagagagacagagaaagatccaatcaattagtggatggattcttagcaattatctgggtcgacctgcggaacctgtgcctcttcagctaccaccgcttgagagacttactcttgattgcaacgaggattgtggaacttctgggacgcagggggtgggaagccctcaaatattggtggaatctcctgcagtattggagtcaggaactgaagaatagtgctgttagcttgcttaatgccatagccatagcagtagctgaggggacagatagaattatagaagtagtacaaaggggggttagagctgttcttaacatacccacaagaataagacagggagcggaaaggcttcttgtataagatgggtggcaagttgtcaaaaagtaagatgcctggatggtctactataagggaaagaatgagacgagctcagccagcagcagagccagcagcagttggggtgggagcagcatctcgagacctggaaagacatggagcactcacaagtagcaatacagcagctaacaatgctgattgtgcctggctagaagcacaagaggacgaggaagtgggttttccagtcagacctcaggtacctcttaggccaatgacttacaagggagctgtagatcttagccactttttaaaagaaaaggggggactggaagggttagtttactcccaaaaaagacaagacatccttgatctgtgggtctaccacacacaaggctacttccctgattggcagaactacacaccagggccagggatcagatatcccctgacctttggatggtgcttcaagctagtaccagttgatccagataaggtagaagaggccaatgaaggagagaacaactgcttattacaccctatggcccagcatgggatggatgacccagagaaagaagtgttagtgtggaagtttgacagccgcctagcatttcatcacatggcccgagagctgcatccggagtactacaaagactgctgagcggccgccctgcaggtcgacctcgagggggggcccggtaccttaattaattaaggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaag

SEQ ID NO: 2 - DNA, 408 bp, Artificial Sequence
  CAEV Promoter
gaattcgtcaactgtgagacatgggctaaagaggactaataacaagctaggccaaattcctgtaaatcacttggggggttataagaaaagcaagttcactatgacaaagcaaaatgtaaaggccaaattcctgtaaatcacttggggggttataagaaaagcaagttcactatgacaaagcaaaatgtaaccgcaagtgctgacagatgtaacagctgacatatcagctgatgcttgctcatgctgacactgtagctctgagctgtatataaggagaagcttgctgcttgcacttcagagttctaggagagtccctcctagtctctcctctccgaggaggtaccgagacctcaaaataaaggagtgattgccttactgccgagtggagagtgattactgagcggcgcc

SEQ ID NO: 3 - DNA, 481 bp, Simian virus 40
tcgagggggggcccggtaccttaattaattaaggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaa

SEQ ID NO: 4 - DNA, 411 bp, Human immunodeficiency virus
aacagcagggactttccacaaggggatgttacggggaggtactggggaggagccggtcgggaacgcccactttcttgatgtataaatatcactgcatttcgctctgtattcagtcgctctgcggagaggctggcaggttgagccctgggaggttctctccagcactagcaggtagagcctgggtgttccctgctagactctcaccagcacttggccggtgctgggcagagtgattccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagca

SEQ ID NO: 5 - DNA, 4981 bp, Simian immunodeficiency virus
cgcccgaacagggacttgaaggagagtgagagactcctgagtacggctgagtgaaggcagtaagggcggcaggaaccaaccacgacggagtgctcctataaaggcgcgggtcggtaccagacggcgtgaggagcgggagaggaagaggcctccggttgcaggtgagtgcaacacaaaaaagaaatagctgtcttttatccaggaaggggtaataagatagagtgggagatgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaataggaacaacagaaactatgccaaaaacaagtagaccaacagcaccatctagcggcagaggaggaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgcctatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtagtcactgctcatattgaaggacagcctgtagaagtattactggatacaggggctgatgattctattgtaacaggaatagagttaggtccacattataccccaaaaatagtaggaggaataggaggttttattaatactaaagaatacaaaaatgtagaaatagaagttttaggcaaaaggattaaagggacaatcatgacaggggacaccccgattaacatttttggtagaaatttgctaacagctctggggatgtctctaaattttcccatagctaaagtagagcctgtaaaagtcgccttaaagccaggaaagaatggaccaaaattgaagcagtggccattatcaaaagaaaagatagttgcattaagagaaatctgggaaaagatggaaaaggatggtcagttggaggaagctcccccgaccaatccatacaacacccccacatttgctataaagaaaaaggataagaacaaatggagaatgctgatagattttagggaactaaatagggtcactcaggactttacggaagtccaattaggaataccacaccctgcaggattagcaaaaaggaaaagaattacagtactggatataggtgatgcatatttctccatacctctagatgaagaatttaggcagtacactgcctttactttaccatcagtaaataatgcagagccaggaaaacgatacatttataaggttctgcctcagggatggaaggggtcaccagccatcttccaatacactatgagacatgtgctagaacccttcaggaaggcaaatccagatgtgaccttagtccagtatatggatgacatcttaatagctagtgacaggacagacctggaacatgacagggtagttttacagtcaaaggaactcttgaatagcatagggttttctaccccagaagagaaattccaaaaagatcccccatttcaatggatggggtacgaattgtggccaacaaaatggaagttgcaaaagatagagttgccacaaagagagacctggacagtgaatgatatacagaagttagtaggagtattaaattgggcagctcaaatttatccaggtataaaaaccaaacatctctgtaggttaattagaggaaaaatgactctaacagaggaagttcagtggactgagatggcagaagcagaatatgaggaaaataaaataattctcagtcaggaacaagaaggatgttattaccaagaaggcaagccattagaagccacggtaataaagagtcaggacaatcagtggtcttataaaattcaccaagaagacaaaatactgaaagtaggaaaatttgcaaagataaagaatacacataccaatggagtgagactattagcacatgtaatacagaaaataggaaaggaagcaatagtgatctggggacaggtcccaaaattccacttaccagttgagaaggatgtatgggaacagtggtggacagactattggcaggtaacctggataccggaatgggattttatctcaacaccaccgctagtaagattagtcttcaatctagtgaaggaccctatagagggagaagaaacctattatacagatggatcgtgtaataaacagtcaaaagaagggaaagcaggatatatcacagataggggcaaagacaaagtaaaagtgttagaacagactactaatcaacaagcagaattggaagcatttctcatggcattgacagactcagggccaaaggcaaatattatagtagattcacaatatgttatgggaataataacaggatgccctacagaatcagagagcaggctagttaatcaaataatagaagaaatgattaaaaagtcagaaatttatgtagcatgggtaccagcacacaaaggtataggaggaaaccaagaaatagaccacctagttagtcaagggattagacaagttctcttcttggaaaagatagagccagcacaagaagaacatgataaataccatagtaatgtaaaagaattggtattcaaatttggattacccagaatagtggccagacagatagtagacacctgtgataaatgccatcagaaaggagaggctatacatgggcaggtaaattcagatctagggacttggcaaatggattgtacccatctagagggaaaaataatcatagttgcagtacatgtagctagtggattcatagaagcagaggtaattccacaagagacaggaagacagacagcactatttctgttaaaattggcaggcagatggcctattacacatctacacacagataatggtgctaactttgcttcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagagatacaatttcaacaatcaaaaaactcaaaatttaaaaattttcgggtctattacagagaaggcagagatcaactgtggaagggacccggtgagctattgtggaaaggggaaggagcagtcatcttaaaggtagggacagacattaaggtagtacccagaagaaaggctaaaattatcaaagattatggaggaggaaaagaggtggatagcagttcccacatggaggataccggagaggttagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagcaccaggtaccaagcctacagtacttagcactgaaagtag

SEQ ID NO: 6 - DNA, 472 bp, Simian immunodeficiency virus
atggaggaggaaaagaggtggatagcagttcccacatggaggataccggagaggttagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagcaccaggtaccaagcctacagtacttagcactgaaagtagtaagcg

SEQ ID NO: 7 - DNA, 2376 bp, Human immunodeficiency virus
cgcccgaacagggacgcgaaagcgaaagtagaaccagaggagctctctcgacgcaggactcggcttgctgaagcgcgcacggcaagaggcgaggggcggcgactggtgagtacgccatttttgactagcggaggctagaaggagagagatgggtgcgagagcgtcaatattaagcgggggacaattagatagatgggaaaaaattcggttacggccagggggaaagaaaagatataagttaaaacatatagtatgggcaagcagagagctagaacgattcgcagttaaccctggcctgttagaaacagcagaaggctgtagacaaatactgggacagctacaaccatcccttcagacaggatcagaggaacttaaatcattatttaatacaatagctaccctctattgtgtacatcaaagaatagagataaaagacaccaaggaagctttagataagatagaggaagagcaaaacaaaagtaagaaaaaagcacagcaagcagcagctgacacaggaaacagcagcagccaagtcagccaaaattaccctatagtgcagaacgctcagggacaaatggtacatcaggccatatcacctagaactttaaatgcatgggtaaaagtagtagaagaaaaggcttttaacccagaagtaatacccatgtttgcagcattgtcagaaggagccaccccacaagatttaaacaccatgctaaacacagtggggggacatcaagcagccatgcaaatattaaaagagactatcaatgaggaagctgcagaatgggatagattgcatccagtacatgcagggcctattgcaccaggccaaatgagagaaccaaggggaagtgacatagcaggaactactagtacccttcaggaacaaataggatggatgacaaataatccacctatcccagtaggagaaatctataaaaaatggataatcatgggattaaataaaattgtaaggatgtatagccctaccagtattctggacataagacaaggaccaaaggaaccctttagagactatgtagaccggttctataaaactctaagagccgagcaagcttcacaggaagtaaaaaattggatgacagaaaccttgttggtccaaaattcaaaccccgattgtaagactattttaaaagcattaggaccaggagctacactagaagaaatgatgacagcatgccagggagtgggaggacctggccataaagcaagagttttggcagaagcaatgagccaagtaacaaatccaacggccgtgatgatgcagaaaagcaattttaggggccaaagaaaaattgttaagtgttttaattgtggcaaagaagggcacatagccaaaaattgcagggctcctagaaaaaagggctgttggaaatgtggaaaggaaggacaccaaatgaaagattgtactgaaagacaggctaattttttagggaagatctggccttcctacaagggaaggccagggaattttcctcaaagcaggctagaaccaacagccccaccagaagcgagcttcaggtttggggaggagacaacaactccccctcagaagcaggagacgatagacaaggaggtgtatcctttaacctccctcagatcactctttggcaacgacccctcgtcacaataaagataggggggcaactaaaagaagctctattagatacaggagcagatgatacagtgttagaagacatgaatttgccaggaaaatggaaaccaaaaatgatagggggaattggaggatttatcaaagtaaaacagtatgatcagatacccatagaaatctgtggacataaaactataggtacagtattaataggacctacacctgtcaacataattggaaggaatttgttgactcagcttggttgcactttaaattttcccattagtcctattgaaactgtaccagtaaaattaaagccaggaatggatggcccaaaagttaagcaatggccattgacagaagaaaaaataaaagcattaatggagatatgcacagaaatggaaaaggaagggaaaatttcaaaaattgggcctgaaaatccatacaatactccagtgtttgccataaagaaaaaagacagtactaagtggagaaaattagtagatttcagagaacttaataagaaaactcaagacttctgggaggttcaattaggaataccacatcccgcggggttaaaaaagaaaaagtcagtaacagtactggatgtgggtgatgcatacttctcagttcccttagatgaagattttaggaagtatactgcatttaccatacctagtataaacaatgagacatcaggaattagatatcagtacaatgtgcttccacagggatggaaaggg

SEQ ID NO: 8 - DNA, 9994 bp, Simian immunodeficiency virus
tggaagggatttattacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcaggttgagccctgggaggttctctccagcactagcaggtagagcctgggtgttccctgctagactctcaccagcacttggccggtgctgggcagagtgattccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagcagattggcgcccgaacagggacgcgaaagcgaaagtagaaccagaggagctctctcgacgcaggactcggcttgctgaagcgcgcacggcaagaggcgaggggcggcgactggtgagtacgccatttttgactagcggaggctagaaggagagagatgggtgcgagagcgtcaatattaagcgggggacaattagatagatgggaaaaaattcggttacggccagggggaaagaaaagatataagttaaaacatatagtatgggcaagcagagagctagaacgattcgcagttaaccctggcctgttagaaacagcagaaggctgtagacaaatactgggacagctacaaccatcccttcagacaggatcagaggaacttaaatcattatttaatacaatagctaccctctattgtgtacatcaaagaatagagataaaagacaccaaggaagctttagataagatagaggaagagcaaaacaaaagtaagaaaaaagcacagcaagcagcagctgacacaggaaacagcagcagccaagtcagccaaaattaccctatagtgcagaacgctcagggacaaatggtacatcaggccatatcacctagaactttaaatgcatgggtaaaagtagtagaagaaaaggcttttaacccagaagtaatacccatgtttgcagcattgtcagaaggagccaccccacaagatttaaacaccatgctaaacacagtggggggacatcaagcagccatgcaaatattaaaagagactatcaatgaggaagctgcagaatgggatagattgcatccagtacatgcagggcctattgcaccaggccaaatgagagaaccaaggggaagtgacatagcaggaactactagtacccttcaggaacaaataggatggatgacaaataatccacctatcccagtaggagaaatctataaaaaatggataatcatgggattaaataaaattgtaaggatgtatagccctaccagtattctggacataagacaaggaccaaaggaaccctttagagactatgtagaccggttctataaaactctaagagccgagcaagcttcacaggaagtaaaaaattggatgacagaaaccttgttggtccaaaattcaaaccccgattgtaagactattttaaaagcattaggaccaggagctacactagaagaaatgatgacagcatgccagggagtgggaggacctggccataaagcaagagttttggcagaagcaatgagccaagtaacaaatccaacggccgtgatgatgcagaaaagcaattttaggggccaaagaaaaattgttaagtgttttaattgtggcaaagaagggcacatagccaaaaattgcagggctcctagaaaaaagggctgttggaaatgtggaaaggaaggacaccaaatgaaagattgtactgaaagacaggctaattttttagggaagatctggccttcctacaagggaaggccagggaattttcctcaaagcaggctagaaccaacagccccaccagaagcgagcttcaggtttggggaggagacaacaactccccctcagaagcaggagacgatagacaaggaggtgtatcctttaacctccctcagatcactctttggcaacgacccctcgtcacaataaagataggggggcaactaaaagaagctctattagatacaggagcagatgatacagtgttagaagacatgaatttgccaggaaaatggaaaccaaaaatgatagggggaattggaggatttatcaaagtaaaacagtatgatcagatacccatagaaatctgtggacataaaactataggtacagtattaataggacctacacctgtcaacataattggaaggaatttgttgactcagcttggttgcactttaaattttcccattagtcctattgaaactgtaccagtaaaattaaagccaggaatggatggcccaaaagttaagcaatggccattgacagaagaaaaaataaaagcattaatggagatatgcacagaaatggaaaaggaagggaaaatttcaaaaattgggcctgaaaatccatacaatactccagtgtttgccataaagaaaaaagacagtactaagtggagaaaattagtagatttcagagaacttaataagaaaactcaagacttctgggaggttcaattaggaataccacatcccgcggggttaaaaaagaaaaagtcagtaacagtactggatgtgggtgatgcatacttctcagttcccttagatgaagattttaggaagtatactgcatttaccatacctagtataaacaatgagacatcaggaattagatatcagtacaatgtgcttccacagggatggaaagggtcaccatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttttggaagaactgaaagaagaagctttaaaacattttgatcctcgcttgctaactgcccttggtaatcatatctataatcgtcacggagacactctagagggagcaggagaactcattagaatcctccaacgagcgctcttcatgcatttcagaggcggatgcatccactccagaatcggccaacctgagggaggaaatcctctctcagctataccgccctctagaagcattctgtagagcaagaaatggagccagtagatcctagactagagccctggaagcatccaggaagtaagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagagatatcagaattatcagcacttgtggagagagggcatcttgctccttgggatattaatgatatgtagcactgcaggacaattgtgggtcacagtctattatggggtacctgtgtggagagaagcaaccaccactctattttgtgcatcagatgctaaagcatatgatacagaggtgcataatgtctgggccacacatgcctgtgtacccacagaccccagcccacaagaaatggcattggaaaatgtgacagaaaattttgacatgtggaaaaataatatggtagaacagatgcatgaagatataatcagcttatgggatcaaagcctaaagccttgtgtaaaattaactccactatgtgttactttaaattgcactgatgtaaagagaaatgctactagtaacactagtagtagctgggaaaggatggaaccaggagaaataaaaaactgctctttcaatgtcacctcaaatataagagataagatgcggaaagaatatgcactcttttataaacttgatgtaataccaataaataatactagtgataatagtgctaaatatagattgataagttgtaacacctcagtccttacacaagcttgtccaaaaatatcctttgagccaattccaatacattattgtaccccggctggttttgcgcttctgaagtgtaatgataaggagttcaatggaacgggaccatgtaaaaatgtcagcacagtacaatgtacacatggaatcaagccagtagtatcaactcaactgctgttaaatggcagtctatcagaaggaggggttgtaattagatctcaaaatttcacaaacaatgctaaaaccataatagtacagctgaatgaaactgtagaaattaattgtacaaggcccaacaacaatacaagaagaagtataaatataggaccagggagagcattttatgcagcagaacaaataataggagatataagacaagcacattgtaacattagtagagcaaaatggaataacactttaaaactgatagttggaaaattacaagaacaatttgggaagaaaacaataatctttaatcaatcctcaggaggagaccctgagattgtaacacacagttttaattgtggaggggaatttttctactgtgattcaacacaactgtttaacagtacttggacgaatgaaaataacgggtccaacactaaagggaatgacacaatcatactaccatgcagaataaaacaaattgtaaacctgtggcaggaagtaggaaaagcaatgtatgcccctcccatcagaagaccaattagatgctcatcaaatattacagggctgctactaacaagagatggtggtcctaataggacgaacgagacattcagacctggaggaggagatatgagggacaattggagaagtgaattatacaaatataaagtagtaaaaattgaaccattaggagtagcacccaccaaggcaaagagaagagtggtgcaaagagaaaaaagagcagtgggaataggagctctgttccttgggttcttgggaacagcaggaagcactatgggcgcagcgtcactgacgctgacggtacaggccagacaattattgtctggtatagtgcaacagcagaacaatttgctgagagctattgaagcgcaacaacatctgttgcagctcacagtctggggcatcaagcagctccaggcaagagtcctggctgtggaaagatacctaagggatcaacagctcctgggaatttggggttgctctggaaaactcatttgcaccactgctgtgccttggaacactagttggagtaataaatctctagatgacatttggaacaacatgacttggatgcagtgggaaagagaaattgacaattacacaaacacaatatacaccttacttcaggaatcacaactccaacaagaacagaatgaaaaagaactattggaattggataaatgggcaagtttgtggaattggttcgatataacaagttggctgtggtatataaaaatattcataatgatagtaggaggcttgataggtttaagaatagtttttactgtactttctatagtgaatagagttaggaagggatactcaccattatcgttccagacccaccgcccagctccagggggacccgacaggcccgaaggaatcgaagaagaaggtggagagagagacagagaaagatccaatcaattagtggatggattcttagcaattatctgggtcgacctgcggaacctgtgcctcttcagctaccaccgcttgagagacttactcttgattgcaacgaggattgtggaacttctgggacgcagggggtgggaagccctcaaatattggtggaatctcctgcagtattggagtcaggaactgaagaatagtgctgttagcttgcttaatgccatagccatagcagtagctgaggggacagatagaattatagaagtagtacaaaggggggttagagctgttcttaacatacccacaagaataagacagggagcggaaaggcttcttgtataagatgggtggcaagttgtcaaaaagtaagatgcctggatggtctactataagggaaagaatgagacgagctcagccagcagcagagccagcagcagttggggtgggagcagcatctcgagacctggaaagacatggagcactcacaagtagcaatacagcagctaacaatgctgattgtgcctggctagaagcacaagaggacgaggaagtgggttttccagtcagacctcaggtacctcttaggccaatgacttacaagggagctgtagatcttagccactttttaaaagaaaaggggggactggaagggttagtttactcccaaaaaagacaagacatccttgatctgtgggtctaccacacacaaggctacttccctgattggcagaactacacaccagggccagggatcagatatcccctgacctttggatggtgcttcaagctagtaccagttgatccagataaggtagaagaggccaatgaaggagagaacaactgcttattacaccctatggcccagcatgggatggatgacccagagaaagaagtgttagtgtggaagtttgacagccgcctagcatttcatcacatggcccgagagctgcatccggagtactacaaagactgctgagcggccgccctgcaggtcgacctcgagggggggcccggtaccttaattaattaaggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattc

SEQ ID NO: 9 - DNA, 10340 bp, Artificial Sequence
  Simian-Human immunodeficiency virus
gaattcactgtgagacatgggctaaagaggactaataacaagctaggccaaattcctgtaaatcacttggggggttataagaaaagcaagttcactatgacaaagcaaaatgtaaaggccaaattcctgtaaatcacttggggggttataagaaaagcaagttcactatgacaaagcaaaatgtaaccgcaagtgctgacagatgtaacagctgacatatcagctgatgcttgctcatgctgacactgtagctctgagctgtatataaggagaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctggcgcccgaacagggacttgaaggagagtgagagactcctgagtacggctgagtgaaggcagtaagggcggcaggaaccaaccacgacggagtgctcctataaaggcgcgggtcggtaccagacggcgtgaggagcgggagaggaagaggcctccggttgcaggtgagtgcaacacaaaaaagaaatagctgtcttttatccaggaaggggtaataagatagagtgggagatgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaataggaacaacagaaactatgccaaaaacaagtagaccaacagcaccatctagcggcagaggaggaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgcctatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtagtcactgctcatattgaaggacagcctgtagaagtattactggatacaggggctgatgattctattgtaacaggaatagagttaggtccacattataccccaaaaatagtaggaggaataggaggttttattaatactaaagaatacaaaaatgtagaaatagaagttttaggcaaaaggattaaagggacaatcatgacaggggacaccccgattaacatttttggtagaaatttgctaacagctctggggatgtctctaaattttcccatagctaaagtagagcctgtaaaagtcgccttaaagccaggaaagaatggaccaaaattgaagcagtggccattatcaaaagaaaagatagttgcattaagagaaatctgggaaaagatggaaaaggatggtcagttggaggaagctcccccgaccaatccatacaacacccccacatttgctataaagaaaaaggataagaacaaatggagaatgctgatagattttagggaactaaatagggtcactcaggactttacggaagtccaattaggaataccacaccctgcaggattagcaaaaaggaaaagaattacagtactggatataggtgatgcatatttctccatacctctagatgaagaatttaggcagtacactgcctttactttaccatcagtaaataatgcagagccaggaaaacgatacatttataaggttctgcctcagggatggaaggggtcaccagccatcttccaatacactatgagacatgtgctagaacccttcaggaaggcaaatccagatgtgaccttagtccagtatatggatgacatcttaatagctagtgacaggacagacctggaacatgacagggtagttttacagtcaaaggaactcttgaatagcatagggttttctaccccagaagagaaattccaaaaagatcccccatttcaatggatggggtacgaattgtggccaacaaaatggaagttgcaaaagatagagttgccacaaagagagacctggacagtgaatgatatacagaagttagtaggagtattaaattgggcagctcaaatttatccaggtataaaaaccaaacatctctgtaggttaattagaggaaaaatgactctaacagaggaagttcagtggactgagatggcagaagcagaatatgaggaaaataaaataattctcagtcaggaacaagaaggatgttattaccaagaaggcaagccattagaagccacggtaataaagagtcaggacaatcagtggtcttataaaattcaccaagaagacaaaatactgaaagtaggaaaatttgcaaagataaagaatacacataccaatggagtgagactattagcacatgtaatacagaaaataggaaaggaagcaatagtgatctggggacaggtcccaaaattccacttaccagttgagaaggatgtatgggaacagtggtggacagactattggcaggtaacctggataccggaatgggattttatctcaacaccaccgctagtaagattagtcttcaatctagtgaaggaccctatagagggagaagaaacctattatacagatggatcgtgtaataaacagtcaaaagaagggaaagcaggatatatcacagataggggcaaagacaaagtaaaagtgttagaacagactactaatcaacaagcagaattggaagcatttctcatggcattgacagactcagggccaaaggcaaatattatagtagattcacaatatgttatgggaataataacaggatgccctacagaatcagagagcaggctagttaatcaaataatagaagaaatgattaaaaagtcagaaatttatgtagcatgggtaccagcacacaaaggtataggaggaaaccaagaaatagaccacctagttagtcaagggattagacaagttctcttcttggaaaagatagagccagcacaagaagaacatgataaataccatagtaatgtaaaagaattggtattcaaatttggattacccagaatagtggccagacagatagtagacacctgtgataaatgccatcagaaaggagaggctatacatgggcaggtaaattcagatctagggacttggcaaatggattgtacccatctagagggaaaaataatcatagttgcagtacatgtagctagtggattcatagaagcagaggtaattccacaagagacaggaagacagacagcactatttctgttaaaattggcaggcagatggcctattacacatctacacacagataatggtgctaactttgcttcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagagatacaatttcaacaatcaaaaaactcaaaatttaaaaattttcgggtctattacagagaaggcagagatcaactgtggaagggacccggtgagctattgtggaaaggggaaggagcagtcatcttaaaggtagggacagacattaaggtagtacccagaagaaaggctaaaattatcaaagattatggaggaggaaaagaggtggatagcagttcccacatggaggataccggagaggttagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagcaccaggtaccaagcctacagtacttagcactgaaagtagtaagcgatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttttggaagaactgaaagaagaagctttaaaacattttgatcctcgcttgctaactgcccttggtaatcatatctataatcgtcacggagacactctagagggagcaggagaactcattagaatcctccaacgagcgctcttcatgcatttcagaggcggatgcatccactccagaatcggccaacctgggggaggaaatcctctctcagctataccgccctctagaagcatgctgtagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaacgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggcacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaattgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctatacgttgacaagttgtaacacctcagtcatttcacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcatggacaatgctaaaaccataatagtacagctgaacacatctgtagaaattaattgtacaagacccagcaacaatacaataaaaagaatccgtatccagagaggaccagggagagcatttgttacaatgggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtggcacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaagggcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacatgacctggatggagtgggacagagaaattaacaattacacaagcttaatacactccttaattgaagaatcgcaaaaccagcaagaaaagaatgaacaagaattattggaattagataaatgggcaagtttgtggaattggtttgacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctacggagcctgtgcctcttcagctaccaccgcttgagagacttactcttgattgtaacgaggactgtggaacttctgggacgcagggggtgggaagccctcaaatattggtggaatctcctacagtattggagtcaggaactaaagaatagtgctgttagcttgctcaatgccatagccatagcagtagctgagggaacagatagggttatagaagtagtccaaggagcttgtagagctattcgctacatacctagaagaataagacagggcttggaaaggattttgctataagattcgagatgggtggagctatttccatgaggcggtccaggcagtctagagatctgcgacagagactcttgcgggcgcgtggggagacttatgggagactcttagaagaggtggaagatggatactcgcgatccccaggaggattagacaagggcttgagctcactctcttgtgagggacagaaatacaatcagggacagtatatgaatactccatggagagacccagctgaagagagagaaaaattagcatacagaaaacaaaatatggatgatatagatgaggaagatgataacttggtaggggtatcagtgaggccaagagttcccctaagaacaatgagttacaaattggcaatagacatgtctcattttataaaagaaaagggggaactggaagggatcttttacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgagcggccgcactgtgagacatgggctaaagaggactaataacaagctaggccaaattcctgtaaatcacttggggggttataagaaaagcaagttcactatgacaaagcaaaatgtaaaggccaaattcctgtaaatcacttggggggttataagaaaagcaagttcactatgacaaagcaaaatgtaaccgcaagtgctgacagatgtaacagctgacatatcagctgatgcttgctcatgctgacactgtagctctgagctgtatataaggagaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctgtcgacctcgagggggg

SEQ ID NO: 10 - DNA, 10449 bp, Artificial Sequence
  Simian-human immunodeficiency virus
gaattcactgtgagacatgggctaaagaggactaataacaagctaggccaaattcctgtaaatcacttggggggttataagaaaagcaagttcactatgacaaagcaaaatgtaaaggccaaattcctgtaaatcacttggggggttataagaaaagcaagttcactatgacaaagcaaaatgtaaccgcaagtgctgacagatgtaacagctgacatatcagctgatgcttgctcatgctgacactgtagctctgagctgtatataaggagaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctggcgcccgaacagggacttgaaggagagtgagagactcctgagtacggctgagtgaaggcagtaagggcggcaggaaccaaccacgacggagtgctcctataaaggcgcgggtcggtaccagacggcgtgaggagcgggagaggaagaggcctccggttgcaggtgagtgcaacacaaaaaagaaatagctgtcttttatccaggaaggggtaataagatagagtgggagatgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaataggaacaacagaaactatgccaaaaacaagtagaccaacagcaccatctagcggcagaggaggaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgcctatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtagtcactgctcatattgaaggacagcctgtagaagtattactggatacaggggctgatgattctattgtaacaggaatagagttaggtccacattataccccaaaaatagtaggaggaataggaggttttattaatactaaagaatacaaaaatgtagaaatagaagttttaggcaaaaggattaaagggacaatcatgacaggggacaccccgattaacatttttggtagaaatttgctaacagctctggggatgtctctaaattttcccatagctaaagtagagcctgtaaaagtcgccttaaagccaggaaaggatggaccaaaattgaagcagtggccattatcaaaagaaaagatagttgcattaagagaaatctgtgaaaagatggaaaaggatggtcagttggaggaagctcccccgaccaatccatacaacacccccacatttgctataaagaaaaaggataagaacaaatggagaatgctgatagattttagggaactaaatagggtcactcaggactttacggaagtccaattaggaataccacaccctgcaggactagcaaaaaggaaaagaattacagtactggatataggtgatgcatatttctccatacctctagatgaagaatttaggcagtacactgcctttactttaccatcagtaaataatgcagagccaggaaaacgatacatttataaggttctgcctcagggatggaaggggtcaccagccatcttccaatacactatgagacatgtgctagaacccttcaggaaggcaaatccagatgtgaccttagtccagtatatggatgacatcttaatagctagtgacaggacagacctggaacatgacagggtagttttacagtcaaaggaactcttgaatagcatagggttttctaccccagaagagaaattccaaaaagatcccccatttcaatggatggggtacgaattgtggccaacaaaatggaagttgcaaaagatagagttgccacaaagagagacctggacagtgaatgatatacagaagttagtaggagtattaaattgggcagctcaaatttatccaggtataaaaaccaaacatctctgtaggttaattagaggaaaaatgactctaacagaggaagttcagtggactgagatggcagaagcagaatatgaggaaaataaaataattctcagtcaggaacaagaaggatgttattaccaagaaggcaagccattagaagccacggtaataaagagtcaggacaatcagtggtcttataaaattcaccaagaagacaaaatactgaaagtaggaaaatttgcaaagataaagaatacacataccaatggagtgagactattagcacatgtaatacagaaaataggaaaggaagcaatagtgatctggggacaggtcccaaaattccacttaccagttgagaaggatgtatgggaacagtggtggacagactattggcaggtaacctggataccggaatgggattttatctcaacaccaccgctagtaagattagtcttcaatctagtgaaggaccctatagagggagaagaaacctattatacagatggatcatgtaataaacagtcaaaagaagggaaagcaggatatatcacagataggggcaaagacaaagtaaaagtgttagaacagactactaatcaacaagcagaattggaagcatttctcatggcattgacagactcagggccaaaggcaaatattatagtagattcacaatatgttatgggaataataacaggatgccctacagaatcagagagcaggctagttaatcaaataatagaagaaatgattaaaaagtcagaaatttatgtagcatgggtaccagcacacaaaggtataggaggaaaccaagaaatagaccacctagttagtcaagggattagacaagttctcttcttggaaaagatagagccagcacaagaagaacatgataaataccatagtaatgtaaaagaattggtattcaaatttggattacccagaatagtggccagacagatagtagacacctgtgataaatgtcatcagaaaggagaggctatacatgggcaggcaaattcagatctagggacttggcaaatggattgtacccatctagagggaaaaataatcatagttgcagtacatgtagctagtggattcatagaagcagaggtaattccacaagagacaggaagacagacagcactatttctgttaaaattggcaggcagatggcctattacacatctacacacagataatggtgctaactttgcttcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagagatacaatttcaacaatcaaaaaactcaaaatttaaaaattttcgggtctattacagagaaggcagagatcaactgtggaagggacccggtgagctattgtggaaaggggaggagcagtcatcttaaaggtagggacagacattaaggtagtacccagaagaaaggctaaaattatcaaagattatggaggaggaaaagaggtggatagcagttcccacatggaggataccggagaggctagagaggtggcatagtagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagcaccaggtaccaagcctacagtacttagcactgaaagtagtaagcgatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttttggaagaactgaaagaagaagctttaaaacattttgatcctcgcttgctaactgcccttggtaatcatatctataatcgtcacggagacactctagagggagcaggagaactcattagaatcctccaacgagcgctcttcatgcatttcagaggcggatgcatccactccagaatcggccaacctgggggaggaaatcctctctcagctataccgccctctagaagcatgctgtagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaacgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggcacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaattgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctatacgttgacaagttgtaacacctcagtcatttcacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcatggacaatgctaaaaccataatagtacagctgaacacatctgtagaaattaattgtacaagacccagcaacaatacaataaaaagaatccgtatccagagaggaccagggagagcatttgttacaatgggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtggcacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaagggcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacatgacctggatggagtgggacagagaaattaacaattacacaagcttaatacactccttaattgaagaatcgcaaaaccagcaagaaaagaatgaacaagaattattggaattagataaatgggcaagtttgtggaattggtttgacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctacggagcctgtgcctcttcagctaccaccgcttgagagacttactcttgattgtaacgaggactgtggaacttctgggacgcagggggtgggaagccctcaaatattggtggaatctcctacagtattggagtcaggaactaaagaatagtgctgttagcttgctcaatgccatagccatagcagtagctgagggaacagatagggttatagaagtagtccaaggagcttgtagagctattcgctacatacctagaagaataagacagggcttggaaaggattttgctataagattcgagatgggtggagctatttccatgaggcggtccaggcagtctagagatctgcgacagagactcttgcgggcgcgtggggagacttatgggagactcttagaagaggtggaagatggatactcgcgatccccaggaggattagacaagggcttgagctcactctcttgtgagggacagaaatacaatcagggacagtatatgaatactccatggagagacccagctgaagagagagaaaaattagcatacagaaaacaaaatatggatgatatagatgaggaagatgataacttggtaggggtatcagtgaggccaagagttcccctaagaacaatgagttacaaattggcaatagacatgtctcattttataaaagaaaagggggaactggaagggatcttttacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgagcggccgcactgtgagacatgggctaaagaggactaataacaagctaggccaaattcctgtaaatcacttggggggttataagaaaagcaagttcactatgacaaagcaaaatgtaaaggccaaattcctgtaaatcacttggggggttataagaaaagcaagttcactatgacaaagcaaaatgtaaccgcaagtgctgacagatgtaacagctgacatatcagctgatgcttgctcatgctgacactgtagctctgagctgtatataaggagaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctgtcgacctcgagggggggcccggtaccttaattaattaaggtaccaggtaagtgtacccaattcgccctatagtgagtcgtattacaattcactcgatcgcccttcccaacagttgcgcagcct

SEQ ID NO: 11 - DNA, 9702 bp, Simian immunodeficiency virus
aattcactgtgagacatgggctaaagaggactaataacaagctaggccaaattcctgtaaatcacttggggggttataagaaaagcaagttcactatgacaaagcaaaatgtaaaggccaaattcctgtaaatcacttggggggttataagaaaagcaagttcactatgacaaagcaaaatgtaaccgcaagtgctgacagatgtaacagctgacatatcagctgatgcttgctcatgctgacactgtagctctgagctgtatataaggagaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctgggcgcctgaacagggacttgaaggagagtgagagactcctgagtacggctgagtgaaggcagtaagggcggcaggaaccaaccacgacggagtgctcctataaaggcgcgggtcggtaccagacggcgtgaggagcgggagaggaagaggcctccggttgcaggtaagtgcaacacaaaaaagaaatagctgtcttttatccaggaaggggtaataagatagagtgggagatgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaacaggaacaacagaaactatgccaaaaacaagtagaccaacagcaccatctagcggcagaggaggaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgccaatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtagtcactgctcatattgaaggacagcctgtagaagtattactggatacaggggctgatgattctattgtaacaggaatagagttaggtccacattataccccaaaaatagtaggaggaataggaggttttattaatactaaagaatacaaaaatgtagaaatagaagttttaggcaaaaggattaaagggacaatcatgacaggggacaccccgattaacatttttggtagaaatttgctaacagctctggggatgtctctaaattttcccatagctaaagtagagcctgtaaaagtcgccttaaagccaggaaaggatggaccaaaattgaagcagtggccattatcaaaagaaaagatagttgcattaagagaaatctgtgaaaagatggaaaaggatggtcagttggaggaagctcccccgaccaatccatacaacacccccacatttgctataaagaaaaaggataagaacaaatggagaatgctgatagattttagggaactaaatagggtcactcaggactttacggaagtccaattaggaataccacaccctgcaggactagcaaaaaggaaaagaattacagtactggatataggtgatgcatatttctccatacctctagatgaagaatttaggcagtacactgcctttactttaccatcagtaaataatgcagagccaggaaaacgatacatttataaggttctgcctcagggatggaaggggtcaccagccatcttccaatacactatgagacatgtgctagaacccttcaggaaggcaaatccagatgtgaccttagtccagtatatggatgacatcttaatagctagtgacaggacagacctggaacatgacagggtagttttacagtcaaaggaactcttgaatagcatagggttttctaccccagaagagaaattccaaaaagatcccccatttcaatggatggggtacgaattgtggccaacaaaatggaagttgcaaaagatagagttgccacaaagagagacctggacagtgaatgatatacagaagttagtaggagtattaaattgggcagctcaaatttatccaggtataaaaaccaaacatctctgtaggttaattagaggaaaaatgactctaacagaggaagttcagtggactgagatggcagaagcagaatatgaggaaaataaaataattctcagtcaggaacaagaaggatgttattaccaagaaggcaagccattagaagccacggtaataaagagtcaggacaatcagtggtcttataaaattcaccaagaagacaaaatactgaaagtaggaaaatttgcaaagataaagaatacacataccaatggagtgagactattagcacatgtaatacagaaaataggaaaggaagcaatagtgatctggggacaggtcccaaaattccacttaccagttgagaaggatgtatgggaacagtggtggacagactattggcaggtaacctggataccggaatgggattttatctcaacaccaccgctagtaagattagtcttcaatctagtgaaggaccctatagagggagaagaaacctattatacagatggatcatgtaataaacagtcaaaagaagggaaagcaggatatatcacagataggggcaaagacaaagtaaaagtgttagaacagactactaatcaacaagcagaattggaagcatttctcatggcattgacagactcagggccaaaggcaaatattatagtagattcacaatatgttatgggaataataacaggatgccctacagaatcagagagcaggctagttaatcaaataatagaagaaatgattaaaaagtcagaaatttatgtagcatgggtaccagcacacaaaggtataggaggaaaccaagaaatagaccacctagttagtcaagggattagacaagttctcttcttggaaaagatagagacagcactatttctgttaaaattggcaggcagatggcctattacacatctacacacagataatggtgctaactttgcttcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagagatacaatttcaacaatcaaaaaactcaaaatttaaaaattttcgggtctattacagagaaggcagagatcaactgtggaagggacccggtgagctattgtggaaaggggaaggagcagtcatcttaaaggtagggacagacattaaggtagtacccagaagaaaggctaaaattatcaaagattatggaggaggaaaagaggtggatagcagttcccacatggaggataccggagaggctagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagtaccaggtaccaagcctacagtacttagcactgaaagtagtaagcgatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttctggaagaactgaaagaagaagctttaaaacattttgatcctcgcttgctaactgcacttggtaatcatatctataatagacatggagacacccttgagggagcaggagaactcattagaatcctccaacgagcgctcttcatgcatttcagaggcggatgcatccactccagaatcggccaacctgggggaggaaatcctctctcagctataccgccctctagaagcatgctataacacatgctattgtaaaaagtgttgctaccattgccagttttgttttcttaaaaaaggcttggggatatgttatgagcaatcacgaaagagaagaagaactccgaaaaaggctaaggctaatacatcttctgcatcaaacaagtaagtatgggatgtcttgggaatcagctgcttatcgccatcttgcttttaagtgtctatgggatctattgtactctatatgtcacagtcttttatggtgtaccagcttggaggaatgcgacaattcccctcttttgtgcaaccaagaatagggatacttggggaacaactcagtgcctaccagataatggtgattattcagaagtggcccttaatgttacagaaagctttgatgcctggaataatacagtcacagaacaggcaatagaggatgtatggcaactctttgagacctcaataaagccttgtgtaaaattatccccattatgcattactatgagatgcaataaaagtgagacagatagatggggattgacaaaatcaataacaacaacagcatcaacaacatcaacgacagcatcagcaaaagtagacatggtcaatgagactagttcttgtatagcccaggataattgcacaggcttggaacaagagcaaatgataagctgtaaattcaacatgacagggttaaaaagagacaagaaaaaagagtacaatgaaacttggtactctgcagatttggtatgtgaacaagggaataacactggtaatgaaagtagatgttacatgaaccactgtaacacttctgttatccaagagtcttgtgacaaacattattgggatgctattagatttaggtattgtgcacctccaggttatgctttgcttagatgtaatgacacaaattattcaggctttatgcctaaatgttctaaggtggtggtctcttcatgcacaaggatgatggagacacagacttctacttggtttggctttaatggaactagagcagaaaatagaacttatatttactggcatggtagggataataggactataattagtttaaataagtattataatctaacaatgaaatgtagaagaccaggaaataagacagttttaccagtcaccattatgtctggattggttttccactcacaaccaatcaatgataggccaaagcaggcatggtgttggtttggaggaaaatggaaggatgcaataaaagaggtgaagcagaccattgtcaaacatcccaggtatactggaactaacaatactgataaaatcaatttgacggctcctggaggaggagatccggaagttaccttcatgtggacaaattgcagaggagagttcctctactgtaaaatgaattggtttctaaattgggtagaagataggaatacagctaaccagaagccaaaggaacagcataaaaggaattacgtgccatgtcatattagacaaataatcaacacttggcataaagtaggcaaaaatgtttatttgcctccaagagagggagacctcacgtgtaactccacagtgaccagtctcatagcaaacatagattggattgatggaaaccaaactaatatcaccatgagtgcagaggtggcagaactgtatcgattggaattgggagattataaattagtagagatcactccaattggcttggcccccacagatgtgaagaggtacactactggtggcacctcaagaaataaaagaggggtctttgtgctagggttcttgggttttctcgcaacggcaggttctgcaatgggcgcggcgtcgttgacgctgaccgctcagtcccgaactttattggctgggatagtgcagcaacagcaacagctgttggacgtggtcaagagacaacaagaattgttgcgactgaccgtctggggaacaaagaacctccagactagggtcactgccatcgagaagtacttaaaggaccaggcgcagctgaatgcttggggatgtgcgtttagacaagtctgccacactactgtaccatggccaaatgcaagtctaacaccaaagtggaacaatgagacttggcaagagtgggagcgaaaggttgacttcttggaagaaaatataacagccctcctagaggaggcacaaattcaacaagagaagaacatgtatgaattacaaaagttgaatagctgggatgtgtttggcaattggtttgaccttgcttcttggataaagtatatacaatatggagtttatatagttgtaggagtaatactgttaagaatagtgatctatatagtacaaatgctagctaagttaaggcaggggtataggccagtgttctcttccccaccctcttatttccagcagacccatatccaacaggacccggcactgccaaccagagaaggcaaagaaagagacggtggagaaggcggtggcaacagctcctggccttggcagatagaatatattcatttcctgatccgccaactgatacgcctcttgacttggctattcagcaactgcagaaccttgctatcgagagtataccagatcctccaaccaatactccagaggctctctgcgaccctacagaggattcgagaagtcctcaggactgaactgacctacctacaatatgggtggagctatttccatgaggcggtccaggccgtctggagatctgcgacagagactcttgcgggcgcgtggggagacttatgggagactcttaggagaggtggaagatggatactcgcaatccccaggaggattagacaagggcttgagctcactctcttgtgagggacagaaatacaatcagggacagtatatgaatactccatggagaaacccagctgaagagagagaaaaattagcatacagaaaacaaaatatggatgatatagatgagtaagatgatgacttggtaggggtatcagtgaggccaaaagttcccctaagaacaatgagttacaaattggcaatagacatgtctcattttataaaagaaaaggggggactggaagggatttattacagtgcaagaagacatagaatcttagacatatacttagaaaaggaagaaggcatcataccagattggcaggattacacctcaggaccaggaattagatacccaaagacatttggctggctatggaaattagtccctgtaaatgtatcagatgaggcacaggaggatgaggagcattatttaatgcatccagctcaaacttcccagtgggatgacccttggggagaggttctagcatggaagtttgatccaactctggcctacacttatgaggcatatgttagatacccagaagagtttggaagcaagtcaggcctgtcagaggaagaggttagaagaaggctaaccgcaagaggccttcttaacatggctgacaagaaggaaactcgctgaaacagcagggacaattcactgtgagacatgggctaaagaggactaataacaagctaggccaaattcctgtaaatcacttggggggttataagaaaagcaagttcactatgacaaagcaaaatgtaaaggccaaattcctgtaaatcacttggggggttataagaaaagcaagttcactatgacaaagcaaaatgtaaccgcaagtgctgacagatgtaacagctgacatatcagctgatgcttgctcatgctgacactgtagctctgagctgtatataaggagaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctg

SEQ ID NO: 12 - DNA, 12856 bp, Artificial Sequence
  sequence of a chimeric immunogenic molecule sequence comprising regulatory elements of caprine arthritis encephalitis lentivirus (CAL-LTR), Vector pCA-LTR-SHIV-KU2
gaattcactgtgagacatgggctaaagaggactaataacaagctaggccaaattcctgtaaatcacttggggggttataagaaaagcaagttcactatgacaaagcaaaatgtaaaggccaaattcctgtaaatcacttggggggttataagaaaagcaagttcactatgacaaagcaaaatgtaaccgcaagtgctgacagatgtaacagctgacatatcagctgatgcttgctcatgctgacactgtagctctgagctgtatataaggagaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctggcgcccgaacagggacttgaaggagagtgagagactcctgagtacggctgagtgaaggcagtaagggcggcaggaaccaaccacgacggagtgctcctataaaggcgcgggtcggtaccagacggcgtgaggagcgggagaggaagaggcctccggttgcaggtgagtgcaacacaaaaaagaaatagctgtcttttatccaggaaggggtaataagatagagtgggagatgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaataggaacaacagaaactatgccaaaaacaagtagaccaacagcaccatctagcggcagaggaggaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgcctatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtagtcactgctcatattgaaggacagcctgtagaagtattactggatacaggggctgatgattctattgtaacaggaatagagttaggtccacattataccccaaaaatagtaggaggaataggaggttttattaatactaaagaatacaaaaatgtagaaatagaagttttaggcaaaaggattaaagggacaatcatgacaggggacaccccgattaacatttttggtagaaatttgctaacagctctggggatgtctctaaattttcccatagctaaagtagagcctgtaaaagtcgccttaaagccaggaaaggatggaccaaaattgaagcagtggccattatcaaaagaaaagatagttgcattaagagaaatctgtgaaaagatggaaaaggatggtcagttggaggaagctcccccgaccaatccatacaacacccccacatttgctataaagaaaaaggataagaacaaatggagaatgctgatagattttagggaactaaatagggtcactcaggactttacggaagtccaattaggaataccacaccctgcaggactagcaaaaaggaaaagaattacagtactggatataggtgatgcatatttctccatacctctagatgaagaatttaggcagtacactgcctttactttaccatcagtaaataatgcagagccaggaaaacgatacatttataaggttctgcctcagggatggaaggggtcaccagccatcttccaatacactatgagacatgtgctagaacccttcaggaaggcaaatccagatgtgaccttagtccagtatatggatgacatcttaatagctagtgacaggacagacctggaacatgacagggtagttttacagtcaaaggaactcttgaatagcatagggttttctaccccagaagagaaattccaaaaagatcccccatttcaatggatggggtacgaattgtggccaacaaaatggaagttgcaaaagatagagttgccacaaagagagacctggacagtgaatgatatacagaagttagtaggagtattaaattgggcagctcaaatttatccaggtataaaaaccaaacatctctgtaggttaattagaggaaaaatgactctaacagaggaagttcagtggactgagatggcagaagcagaatatgaggaaaataaaataattctcagtcaggaacaagaaggatgttattaccaagaaggcaagccattagaagccacggtaataaagagtcaggacaatcagtggtcttataaaattcaccaagaagacaaaatactgaaagtaggaaaatttgcaaagataaagaatacacataccaatggagtgagactattagcacatgtaatacagaaaataggaaaggaagcaatagtgatctggggacaggtcccaaaattccacttaccagttgagaaggatgtatgggaacagtggtggacagactattggcaggtaacctggataccggaatgggattttatctcaacaccaccgctagtaagattagtcttcaatctagtgaaggaccctatagagggagaagaaacctattatacagatggatcatgtaataaacagtcaaaagaagggaaagcaggatatatcacagataggggcaaagacaaagtaaaagtgttagaacagactactaatcaacaagcagaattggaagcatttctcatggcattgacagactcagggccaaaggcaaatattatagtagattcacaatatgttatgggaataataacaggatgccctacagaatcagagagcaggctagttaatcaaataatagaagaaatgattaaaaagtcagaaatttatgtagcatgggtaccagcacacaaaggtataggaggaaaccaagaaatagaccacctagttagtcaagggattagacaagttctcttcttggaaaagatagagccagcacaagaagaacatgataaataccatagtaatgtaaaagaattggtattcaaatttggattacccagaatagtggccagacagatagtagacacctgtgataaatgtcatcagaaaggagaggctatacatgggcaggcaaattcagatctagggacttggcaaatggattgtacccatctagagggaaaaataatcatagttgcagtacatgtagctagtggattcatagaagcagaggtaattccacaagagacaggaagacagacagcactatttctgttaaaattggcaggcagatggcctattacacatctacacacagataatggtgctaactttgcttcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagagatacaatttcaacaatcaaaaaactcaaaatttaaaaattttcgggtctattacagagaaggcagagatcaactgtggaagggacccggtgagctattgtggaaaggggaggagcagtcatcttaaaggtagggacagacattaaggtagtacccagaagaaaggctaaaattatcaaagattatggaggaggaaaagaggtggatagcagttcccacatggaggataccggagaggctagagaggtggcatagtagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagcaccaggtaccaagcctacagtacttagcactgaaagtagtaagcgatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttttggaagaactgaaagaagaagctttaaaacattttgatcctcgcttgctaactgcccttggtaatcatatctataatcgtcacggagacactctagagggagcaggagaactcattagaatcctccaacgagcgctcttcatgcatttcagaggcggatgcatccactccagaatcggccaacctgggggaggaaatcctctctcagctataccgccctctagaagcatgctgtagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaacgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggcacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaattgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctatacgttgacaagttgtaacacctcagtcatttcacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcatggacaatgctaaaaccataatagtacagctgaacacatctgtagaaattaattgtacaagacccagcaacaatacaataaaaagaatccgtatccagagaggaccagggagagcatttgttacaatgggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtggcacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaagggcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacatgacctggatggagtgggacagagaaattaacaattacacaagcttaatacactccttaattgaagaatcgcaaaaccagcaagaaaagaatgaacaagaattattggaattagataaatgggcaagtttgtggaattggtttgacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctacggagcctgtgcctcttcagctaccaccgcttgagagacttactcttgattgtaacgaggactgtggaacttctgggacgcagggggtgggaagccctcaaatattggtggaatctcctacagtattggagtcaggaactaaagaatagtgctgttagcttgctcaatgccatagccatagcagtagctgagggaacagatagggttatagaagtagtccaaggagcttgtagagctattcgctacatacctagaagaataagacagggcttggaaaggattttgctataagattcgagatgggtggagctatttccatgaggcggtccaggcagtctagagatctgcgacagagactcttgcgggcgcgtggggagacttatgggagactcttagaagaggtggaagatggatactcgcgatccccaggaggattagacaagggcttgagctcactctcttgtgagggacagaaatacaatcagggacagtatatgaatactccatggagagacccagctgaagagagagaaaaattagcatacagaaaacaaaatatggatgatatagatgaggaagatgataacttggtaggggtatcagtgaggccaagagttcccctaagaacaatgagttacaaattggcaatagacatgtctcattttataaaagaaaagggggaactggaagggatcttttacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgagcggccgcactgtgagacatgggctaaagaggactaataacaagctaggccaaattcctgtaaatcacttggggggttataagaaaagcaagttcactatgacaaagcaaaatgtaaaggccaaattcctgtaaatcacttggggggttataagaaaagcaagttcactatgacaaagcaaaatgtaaccgcaagtgctgacagatgtaacagctgacatatcagctgatgcttgctcatgctgacactgtagctctgagctgtatataaggagaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctgtcgacctcgagggggggcccggtaccttaattaattaaggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 13 - DNA, 12854 bp, Artificial Sequence
  sequence of a chimeric immunogenic molecule sequence comprising regulatory elements of caprine arthritis encephalitis lentivirus, Vector pCA-LTR-SHIV-KU2-IN
gaattcactgtgagacatgggctaaagaggactaataacaagctaggccaaattcctgtaaatcacttggggggttataagaaaagcaagttcactatgacaaagcaaaatgtaaaggccaaattcctgtaaatcacttggggggttataagaaaagcaagttcactatgacaaagcaaaatgtaaccgcaagtgctgacagatgtaacagctgacatatcagctgatgcttgctcatgctgacactgtagctctgagctgtatataaggagaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctggcgcccgaacagggacttgaaggagagtgagagactcctgagtacggctgagtgaaggcagtaagggcggcaggaaccaaccacgacggagtgctcctataaaggcgcgggtcggtaccagacggcgtgaggagcgggagaggaagaggcctccggttgcaggtgagtgcaacacaaaaaagaaatagctgtcttttatccaggaaggggtaataagatagagtgggagatgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaataggaacaacagaaactatgccaaaaacaagtagaccaacagcaccatctagcggcagaggaggaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgcctatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtagtcactgctcatattgaaggacagcctgtagaagtattactggatacaggggctgatgattctattgtaacaggaatagagttaggtccacattataccccaaaaatagtaggaggaataggaggttttattaatactaaagaatacaaaaatgtagaaatagaagttttaggcaaaaggattaaagggacaatcatgacaggggacaccccgattaacatttttggtagaaatttgctaacagctctggggatgtctctaaattttcccatagctaaagtagagcctgtaaaagtcgccttaaagccaggaaagaatggaccaaaattgaagcagtggccattatcaaaagaaaagatagttgcattaagagaaatctgggaaaagatggaaaaggatggtcagttggaggaagctcccccgaccaatccatacaacacccccacatttgctataaagaaaaaggataagaacaaatggagaatgctgatagattttagggaactaaatagggtcactcaggactttacggaagtccaattaggaataccacaccctgcaggattagcaaaaaggaaaagaattacagtactggatataggtgatgcatatttctccatacctctagatgaagaatttaggcagtacactgcctttactttaccatcagtaaataatgcagagccaggaaaacgatacatttataaggttctgcctcagggatggaaggggtcaccagccatcttccaatacactatgagacatgtgctagaacccttcaggaaggcaaatccagatgtgaccttagtccagtatatggatgacatcttaatagctagtgacaggacagacctggaacatgacagggtagttttacagtcaaaggaactcttgaatagcatagggttttctaccccagaagagaaattccaaaaagatcccccatttcaatggatggggtacgaattgtggccaacaaaatggaagttgcaaaagatagagttgccacaaagagagacctggacagtgaatgatatacagaagttagtaggagtattaaattgggcagctcaaatttatccaggtataaaaaccaaacatctctgtaggttaattagaggaaaaatgactctaacagaggaagttcagtggactgagatggcagaagcagaatatgaggaaaataaaataattctcagtcaggaacaagaaggatgttattaccaagaaggcaagccattagaagccacggtaataaagagtcaggacaatcagtggtcttataaaattcaccaagaagacaaaatactgaaagtaggaaaatttgcaaagataaagaatacacataccaatggagtgagactattagcacatgtaatacagaaaataggaaaggaagcaatagtgatctggggacaggtcccaaaattccacttaccagttgagaaggatgtatgggaacagtggtggacagactattggcaggtaacctggataccggaatgggattttatctcaacaccaccgctagtaagattagtcttcaatctagtgaaggaccctatagagggagaagaaacctattatacagatggatcgtgtaataaacagtcaaaagaagggaaagcaggatatatcacagataggggcaaagacaaagtaaaagtgttagaacagactactaatcaacaagcagaattggaagcatttctcatggcattgacagactcagggccaaaggcaaatattatagtagattcacaatatgttatgggaataataacaggatgccctacagaatcagagagcaggctagttaatcaaataatagaagaaatgattaaaaagtcagaaatttatgtagcatgggtaccagcacacaaaggtataggaggaaaccaagaaatagaccacctagttagtcaagggattagacaagttctcttcttggaaaagatagagccagcacaagaagaacatgataaataccatagtaatgtaaaagaattggtattcaaatttggattacccagaatagtggccagacagatagtagacacctgtgataaatgccatcagaaaggagaggctatacatgggcaggtaaattcagatctagggacttggcaaatggattgtacccatctagagggaaaaataatcatagttgcagtacatgtagctagtggattcatagaagcagaggtaattccacaagagacaggaagacagacagcactatttctgttaaaattggcaggcagatggcctattacacatctacacacagataatggtgctaactttgcttcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagagatacaatttcaacaatcaaaaaactcaaaatttaaaaattttcgggtctattacagagaaggcagagatcaactgtggaagggacccggtgagctattgtggaaaggggaaggagcagtcatcttaaaggtagggacagacattaaggtagtacccagaagaaaggctaaaattatcaaagattatggaggaggaaaagaggtggatagcagttcccacatggaggataccggagaggttagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagcaccaggtaccaagcctacagtacttagcactgaaagtagtaagcgatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttttggaagaactgaaagaagaagctttaaaacattttgatcctcgcttgctaactgcccttggtaatcatatctataatcgtcacggagacactctagagggagcaggagaactcattagaatcctccaacgagcgctcttcatgcatttcagaggcggatgcatccactccagaatcggccaacctgggggaggaaatcctctctcagctataccgccctctagaagcatgctgtagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaacgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggcacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaattgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctatacgttgacaagttgtaacacctcagtcatttcacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcatggacaatgctaaaaccataatagtacagctgaacacatctgtagaaattaattgtacaagacccagcaacaatacaataaaaagaatccgtatccagagaggaccagggagagcatttgttacaatgggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtggcacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaagggcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacatgacctggatggagtgggacagagaaattaacaattacacaagcttaatacactccttaattgaagaatcgcaaaaccagcaagaaaagaatgaacaagaattattggaattagataaatgggcaagtttgtggaattggtttgacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctacggagcctgtgcctcttcagctaccaccgcttgagagacttactcttgattgtaacgaggactgtggaacttctgggacgcagggggtgggaagccctcaaatattggtggaatctcctacagtattggagtcaggaactaaagaatagtgctgttagcttgctcaatgccatagccatagcagtagctgagggaacagatagggttatagaagtagtccaaggagcttgtagagctattcgctacatacctagaagaataagacagggcttggaaaggattttgctataagattcgagatgggtggagctatttccatgaggcggtccaggcagtctagagatctgcgacagagactcttgcgggcgcgtggggagacttatgggagactcttagaagaggtggaagatggatactcgcgatccccaggaggattagacaagggcttgagctcactctcttgtgagggacagaaatacaatcagggacagtatatgaatactccatggagagacccagctgaagagagagaaaaattagcatacagaaaacaaaatatggatgatatagatgaggaagatgataacttggtaggggtatcagtgaggccaagagttcccctaagaacaatgagttacaaattggcaatagacatgtctcattttataaaagaaaagggggaactggaagggatcttttacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgagcggccgcactgtgagacatgggctaaagaggactaataacaagctaggccaaattcctgtaaatcacttggggggttataagaaaagcaagttcactatgacaaagcaaaatgtaaaggccaaattcctgtaaatcacttggggggttataagaaaagcaagttcactatgacaaagcaaaatgtaaccgcaagtgctgacagatgtaacagctgacatatcagctgatgcttgctcatgctgacactgtagctctgagctgtatataaggagaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctgtcgacctcgagggggggcccggtaccttaattaattaaggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 14 - DNA, 458 bp, Artificial Sequence
  3′LTR of caprine arthritis encephalitis lentivirus
aattcactgtgagacatgggctaaagaggactaataacaagctaggccaaattcctgtaaatcacttggggggttataagaaaagcaagttcactatgacaaagcaaaatgtaaaggccaaattcctgtaaatcacttggggggttataagaaaagcaagttcactatgacaaagcaaaatgtaaccgcaagtgctgacagatgtaacagctgacatatcagctgatgcttgctcatgctgacactgtagctctgagctgtatataaggagaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctgc